(19) United States
(12) Patent Application Publication
(10) Pub. No.: US 2004/0248837 A1
(43) Pub. Date: Dec. 9, 2004

(54) Methods of treating pulmonary fibrotic disorders

(76) Inventors: Eyal Raz, Del Mar, CA (US); David Broide, San Diego, CA (US); Kenji Takabayashi, San Diego, CA (US)

Correspondence Address: BOZICEVIC, FIELD & FRANCIS LLP, 1900 UNIVERSITY AVE, SUITE 200, EAST PALO ALTO, CA, 94303, US

(21) Appl. No.: 10/697,817

(22) Filed: Oct. 29, 2003

Related U.S. Application Data
(60) Provisional application No. 60/423,035, filed on Nov. 1, 2002.

(51) Int. Cl. A61K 48/00

(52) U.S. Cl. 514/44

(57) ABSTRACT

The present invention provides methods of treating airway remodeling, the methods generally involve administering an effective amount of a Toll-like receptor agonist to an individual suffering from airway remodeling. The present invention provides methods of treating pulmonary fibrosis, the methods generally involving administering an effective amount of a Toll-like receptor agonist to an individual in need thereof. The present invention further provides pharmaceutical compositions comprising a TLR agonist and a formulation suitable for delivery by inhalation.

### CROSS-REFERENCE

This application claims the benefit of U.S. Provisional Patent Application No. 60/423,035, filed Nov. 1, 2002, which application is incorporated herein by reference in its entirety.

### STATEMENT REGARDING FEDERALLY SPONSORED RESEARCH

The U.S. government may have certain rights in this invention, pursuant to grant nos. AI40682, AI 33977, and AI 38425 awarded by the National Institutes of Health.

### FIELD OF THE INVENTION

This application is in the field of fibrosis, particularly pulmonary fibrotic disorders, and the use of toll-like receptor ligands to treat fibrotic disorders of the airways and the interstitium.

### BACKGROUND OF THE INVENTION

Interstitial lung disease is a general term that includes a variety of chronic lung disorders. Interstitial lung disease involves lung damage followed by inflammation of the alveoli, and subsequently by fibrosis in the interstitium (the tissue between alveoli). The terms “interstitial lung disease,” “pulmonary fibrosis,” and “interstitial pulmonary fibrosis” are often used to describe the same disorder. Some interstitial lung diseases have known causes, while other interstitial lung diseases, referred to as “idiopathic,” have unknown causes.

Some of the known causes include occupational and environmental exposures (e.g., exposure to silica dust, asbestos fibers, metal dusts, and the like); sarcoidosis; certain drugs; radiation; connective tissue or collagen diseases; and genetic causes. When all known causes of interstitial lung disease have been ruled out, the condition is called “idiopathic pulmonary fibrosis.” Symptoms of interstitial lung disease include shortness of breath, fatigue and weakness, loss of appetite, loss of weight, dry cough that does not produce phlegm, discomfort in the chest, labored breathing, and hemorrhage in the lungs. Treatments of interstitial lung diseases are currently limited to corticosteroids, interferon-gamma, oxygen supplementation from portable containers, and lung transplantation.

Chronic asthma is associated with chronic inflammation of the airways followed by repair. The end result of repeated cycles of inflammation and repair may be imperfect repair resulting in a structurally and functionally abnormal remodeling of the airways. The structural remodeling changes noted in asthmatic airway include subepithelial fibrosis, an increased smooth muscle mass, and an increase in mucous glands. Attempts to study the mechanism and significance of airway remodeling in asthma have been hindered in human asthmatics by the difficulties inherent in prospectively following sufficient numbers of asthmatics with chronic airway inflammation for sufficient time periods to observe whether remodeling of the airways occurs. In addition, most asthmatics are on anti-inflammatory therapy, which may prevent remodeling of the airways. Mouse models of asthma have provided important insight into the mechanism of acute allergen induced airway inflammation and airway hyperreactivity, but have been considered unsuitable for the study of airway remodeling, as recurrent nebulized antigen challenge induces tolerance instead of chronic airway inflammation (which is considered to precede airway remodeling).

There is a need in the art for methods of treating pulmonary fibrotic disorders, including interstitial pulmonary fibrosis, and airway remodeling. The present invention addresses this need.

Literature

Santeliz et al. (2002) J. Allergy Clin. Immunol. 109:455-462; Broide et al. (2001) J. Clin. Immunol. 21:175-182; WO 99/11275; U.S. Pat. No. 6,426,336; Ziesche et al. (1999) N. Engl. J. Med. 341:1264-1269; EP 795,332; Published U.S. Patent Application No. 20030139364; Underhill and Ozinsky (2002) Curr. Opin. Immunol. 14:103-110; Jain et al. (2002) J. Allergy Clin. Immunol. 110:867-872; Dhainaut et al. (2003) Crit. Care Med. 31 (4 Suppl) S258-S264; Munger et al. (1999) Cell 96:319-328; Pittet et al. (2001) J. Clin. Invest. 107:1537-1544; Morris et al. (2003) Nature 422:169-173; Roberts et al. (2003) Nature 422:130-131; Akira (2003) Curr. Opinion Immunol. 15:5-11; Lee et al. (2003) Proc. Natl. Acad. Sci. USA 100:6646-6651; Sabroe et al. (2003) J. Immunol. 171:1630-1635; Akira and Hemmi (2003) Immunol. Lett. 85:85-95; Zuany-Amorim et al. (2002) Nature Reviews 1:797-807; Kaminski et al. (2003) Am. J. Resp. Cell Mol. Biol. 29:S1-S105; Fearson et al. (2003) Eur. J. Immunol. 33:2114-2122; Marshall et al. (2003) J. Leukocyte Biol. 73:781-792; Verthelyi et al. (2001) J. Immunol. 166:2372-2377.

### SUMMARY OF THE INVENTION

The present invention provides methods of treating airway remodeling, the methods generally involve administering an effective amount of a Toll-like receptor agonist to an individual suffering from airway remodeling. The present invention provides methods of treating pulmonary fibrosis, the methods generally involving administering an effective amount of a Toll-like receptor agonist to an individual in need thereof. The present invention further provides pharmaceutical compositions comprising a TLR agonist and a formulation suitable for delivery by inhalation.

Features of the Invention

The present invention features a method for treating pulmonary fibrosis in an individual. Subject methods for inhibiting or reversing pulmonary fibrosis (or “interstitial lung disease”) reduce fibrosis of the lung tissue in the interstitium. The methods generally involve administering to the individual a Toll-like receptor (TLR) agonist in an amount effective to inhibit or reverse a pulmonary fibrosis in the individual.

Pulmonary fibrosis is associated with any of a variety of disorders, including, but not limited to, idiopathic pulmonary fibrosis, interstitial pneumonia, sarcoidosis, chronic obstructive pulmonary disease (COPD), irradiation-induced lung fibrosis, cystic fibrosis, chronic airway exposure to an irritant, chronic viral infection of the airways, chronic mycoplasma infection of the airways, and chronic bacterial infection of the airways. In general, airway fibrosis involves the parenchyma. Thus, the present invention provides a method for treating pulmonary fibrosis (“lung fibrosis”), regardless of the underlying cause.

The present invention provides methods for treating airway remodeling that occurs as a result of chronic asthma. Airway remodeling is frequently associated with chronic asthma. In general, airway remodeling involves deposition of extracellular matrix. Typically, the parenchyma are not involved. The methods generally involve administering to the individual in need thereof a TLR agonist in an amount effective to inhibit or reverse airway remodeling.

In some embodiments, the TLR agonist is a therapeutic nucleic acid that comprises the nucleotide sequence 5′ CG 3′. In some of these embodiments, the therapeutic nucleic acid comprises the nucleotide sequence 5′-purine-purine-cytosine-guanine-pyrimidine-pyrimidine-3′. In other embodiments, the therapeutic nucleic acid comprises the nucleotide sequence 5′-purine-TCG-pyrimidine-pyrimidine-3′. In other embodiments, the therapeutic nucleic acid comprises the nucleotide sequence 5′-(TGC)ₙ-3′, where n≧1. In other embodiments, the therapeutic nucleic acid comprises the sequence 5′-TCGNN-3′, where N is any nucleotide.

In some embodiments, the TLR agonist is administered to the respiratory tract of the individual. In other embodiments, the TLR agonist is administered intranasally. In other embodiments, the TLR agonist is administered systemically.

In some embodiments, the methods further involve administering an effective amount of at least a second therapeutic agent. In some embodiments, the methods further involve administering an effective amount of a bronchodilator. In other embodiments, the methods further involve administering an effective amount of an anti-inflammatory agent. In other embodiments, the methods further involve administering an effective amount of interferon-gamma (IFN-γ).

### BRIEF DESCRIPTION OF THE DRAWINGS

FIG. 1 is a schematic representation of the experimental protocol for generating airway remodeling in a chronic asthma model.

FIG. 2 depicts the effect of ISS on the area of peribronchial trichrome staining.

FIG. 3 depicts the effect of ISS on the level of collagen V staining.

FIG. 4 depicts the effect of ISS on peribronchial smooth muscle layer thickness.

FIG. 5 depicts the effect of ISS on the level of α-smooth muscle actin staining.

FIG. 6 depicts the effect of ISS on mucus production.

FIG. 7 depicts the effect of ISS on Muc5ac mRNA levels.

FIG. 8 depicts induction of enzymatic activity of various MMPs by ISS in lung tissue.

FIG. 9 depicts suppression of integrin β₆ gene transcription by various TLR ligands in lung tissue.

### DEFINITIONS

As used herein, the term “interstitial lung disease” refers to any disease characterized by fibrosis in the interstitium (i.e., the tissue between the alveoli), regardless of the cause. Disorders that can lead to interstitial lung disease include, but are not limited to, connective tissue diseases, such as scleroderma, polymyositis-dermatomyositis, systemic lupus erythematosus, rheumatoid arthritis, ankylosing spondylitis, and mixed connective tissue disease; treatment-induced causes, e.g., treatment with antibiotics (e.g., furantoin, sulfasalazine, etc.), treatment with antiarrhythmic agents (e.g., amiodarone, tocainide, propanolol, etc.), treatment with anti-inflammatory agents (e.g., gold, penicillamine, etc.), treatment with anti-convulsants (e.g., phenyloin), treatment with chemotherapeutic agents (e.g., mitomycin C, bleomycin, busulfan, cyclophosphamide, azathioprine, 1,3-N,N′-bis(2-chloroethyl)-N-nitrosourea (BCNU), methotrexate, etc.), treatment with radiation, treatment with oxygen; drug-induced causes, e.g., cocaine use; sarcoidosis; eosinophilic granuloma; amyloidosis; lymphangitic carcinoma; bronchoalveolar carcinoma; pulmonary lymphoma; Adult Respiratory Distress Syndrome; acquired immunodeficiency syndrome; bone marrow transplantation; chronic obstructive pulmonary disease; cystic fibrosis; any chronic airway inflammatory disorder; viral infection of the lungs (e.g., infection with adenovirus, respiratory syncytial virus, influenza virus, etc.); fungal infection of the lungs; mycoplasma infection (e.g., infection with Mycoplasma pulmonis, M. pneumonia, M. tuberculosis, etc.); bacterial infection of the lungs (e.g., infection with Klebsiella, Staphylococcus aureus, etc.); respiratory bronchiolitis; eosinophilic pneumonia; diffuse alveolar hemorrhage syndrome; disorders resulting from chronic exposure to inorganic dusts, e.g., asbestosis, silicosis, coal worker's pneumoconiosis, and talc pneumoconiosis; disorders resulting from chronic exposure to organic dusts, e.g., bird breeder's lung, farmer's lung; idiopathic pulmonary fibrosis; acute interstitial pneumonia (AIP); usual interstitial pneumonia (UIP), including sporadic form and familial form; desquamative interstitial pneumonia/respiratory bronchiolitis interstitial lung disease; and nonspecific interstitial pneumonia.

As used herein, the terms “treatment”, “treating”, and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. “Treatment,” as used herein, covers any treatment of a disease in a mammal, particularly in a human, and includes: (a) increasing survival time; (b) decreasing the risk of death due to the disease; (c) preventing the disease from occurring in a subject which may be predisposed to the disease but has not yet been diagnosed as having it; (d) inhibiting the disease, i.e., arresting its development (e.g., reducing the rate of disease progression); and (e) relieving the disease, i.e., causing regression of the disease.

The terms “individual,” “host,” “subject,” and “patient,” used interchangeably herein, refer to a mammal, particularly a human.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “a therapeutic nucleic acid” includes a plurality of such nucleic acids and reference to “the TLR agonist” includes reference to one or more TLR agonists and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates, which may need to be independently confirmed.

### DETAILED DESCRIPTION OF THE INVENTION

The present invention provides methods of treating airway remodeling, the methods generally involve administering an effective amount of a Toll-like receptor agonist to an individual suffering from airway remodeling. The present invention provides methods of treating pulmonary fibrosis, the methods generally involving administering an effective amount of a Toll-like receptor agonist to an individual in need thereof. The present invention further provides pharmaceutical compositions comprising a TLR agonist and a formulation suitable for delivery by inhalation.

Treatment Methods

The present invention provides methods of treating airway remodeling, the methods generally involve administering an effective amount of a Toll-like receptor agonist to an individual suffering from airway remodeling. The present invention provides methods of treating pulmonary fibrosis, the methods generally involving administering an effective amount of a Toll-like receptor agonist to an individual in need thereof.

Airway Remodeling

The present invention provides a method of treating airway remodeling in an individual. The methods generally involve administering to an individual in need thereof an effective amount of a TLR agonist to treat the airway remodeling. The airways include the trachea, the bronchi, and the bronchioles.

In some embodiments, an “effective amount” of a TLR agonist is an amount that results in a reduction of at least one pathological parameter associated with airway remodeling. Thus, e.g., in some embodiments, an effective amount of a TLR agonist is an amount that is effective to achieve a reduction of at least about 10%, at least about 15%, at least about 20%, or at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%, compared to the expected reduction in the parameter in an individual having airway remodeling and not treated with the TLR agonist.

Pathological parameters include, but are not limited to, mucus hypersecretion in the lungs; mucous cell hyperplasia; basement membrane thickening; airway nerve fiber rearrangement; individual airway narrowing; increased alveolarization; peribronchial trichrome staining; peribronchial collagen production; smooth muscle layer thickness; peribronchial myoblast α-smooth muscle actin production; smooth muscle hypertrophy, hyperplasia, and differentiation into myoblasts; increased number of peribronchial mast cells; release of inflammatory mediators by peribronchial mast cells; and the like. As one non-limiting example, airway remodeling, which is associated with chronic asthma, is characterized by one or more of the foregoing pathological parameters. The instant invention provides methods of treating airway remodeling that is associated with chronic asthma.

Peribronchial trichrome staining is a measure of the degree of fibrosis. Trichrome staining is carried out using any known method. See, e.g., “Theory and Practice of Histological Techniques” (2002) J. D. Bancroft and M. Gamble, eds., W. B. Saunders Co.; and “Theory and Practice of Histotechnology” (1987) D. C. Sheehan, C. V. Mosby Co. Kits for conducting trichrome staining are commercially available and can be used to determine the degree of fibrosis. The degree of trichrome staining can be expressed, e.g., as the stained area in μm² per μm circumference of bronchiole. A decrease in the area of trichrome staining following treatment with a subject method indicates the efficacy of the method.

Immunohistochemical techniques are useful for determining peribronchial collagen production and peribronchial α-smooth muscle actin production. As a non-limiting example, an antibody to collagen V is used to detect peribronchial collagen in a lung tissue biopsy. As another non-limiting example, an antibody to α-smooth muscle actin is used to detect peribronchial α-smooth muscle actin in a lung biopsy. Immunohistochemical staining is carried out using methods well known in the art. An antibody specific for collagen or for α-smooth muscle actin is detectably labeled, and the antibody is contacted with lung biopsy samples, e.g., as tissue sections. The detectable label is either a direct label or an indirect label. Direct labels include fluorochromes, radiolabels, enzymes that produce a detectable product, and the like. Indirect labels include detectably labeled secondary antibodies, e.g., antibodies that bind to the primary antibody specific for collagen or α-smooth muscle actin. The degree of staining with such antibodies can be expressed, e.g., as the stained area in μm² per μm circumference of bronchiole. A decrease in the area of staining with antibody to α-smooth muscle actin and/or antibody to collagen (e.g., collagen V) following treatment with a subject method indicates the efficacy of the method.

Peribronchial smooth muscle layer thickness is measured in a lung biopsy sample using standard techniques. For example, the thickness of peribronchial smooth muscle layer is measured from the innermost aspect to the outermost aspect of the circumferential smooth muscle layer.

Airway mucus expression is measured using any known method. Typical methods of measuring airway mucus expression include periodic acid Schiffs (PAS) stain; PAS/Alcian blue (PAS/AB) stain; detection of Muc5ac mRNA; and the like. PAS and PAS/AB staining methods are well known in the art. Muc5ac mRNA is detected using any known method, including, but not limited to, a reverse transcription/polymerase chain reaction (RT-PCR) method using primers specific for Muc5ac cDNA; RNA (Northern) blotting using a labeled probe specific for Muc5ac mRNA; and the like.

The number of mast cells in the airways is determined using any known method. Mast cell number can be determined using standard histological evaluation (e.g., hematoxylin-eosin staining; immunohistochemical staining; etc.); determining levels of mast cell growth factors (e.g., IL-9, IL-4, Stem Cell Factor) by ELISA or in situ hybridization; and the like.

Whether a given TLR agonist, alone or in combination therapy, as described below, is effective to treat airway remodeling can be readily determined using standard assays. For example, an animal model of chronic asthma, as described in the Examples, is used to determine whether a given TLR agonist is effective in reducing airway remodeling associated with chronic asthma. In a patient being treated for airway remodeling, any of a variety of tests can be used to determine efficacy. For example, a lung biopsy sample can be analyzed for any of the above-described pathological parameters. In addition, tests to measure lung function, e.g., spirometry, can be used to asses the beneficial effects of a TLR agonist treatment on lung function.

Interstitial Lung Disease

The present invention provides methods of treating interstitial lung disease in an individual in need thereof. The subject methods generally involve administering an effective amount of a TLR agonist to the individual. Administering an effective amount of a TLR agonist accomplishes one or more of the following: 1) reduces a pathological parameter associated with pulmonary fibrosis; 2) increases at least one parameter or measure of lung function; 3) arrest progression of the disease; 4) slows progression of the disorder; 5) increases probability of survival; 6) reduces risk of death due to the disorder or complications of the disorder; 7) reduces the risk that the individual will develop the disorder; and 8) reduces the amount of a therapeutic agent, other than a TLR agonist, that needs to be administered.

In some embodiments, an “effective amount” of a TLR agonist is an amount that results in a reduction of at least one pathological parameter or symptom associated with interstitial lung disease. Thus, e.g., in some embodiments, an effective amount of a TLR agonist is an amount that is effective to achieve a reduction of at least about 10%, at least about 15%, at least about 20%, or at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%, compared to the expected reduction in the parameter or the symptom in an individual having interstitial lung disease and not treated with the TLR agonist.

Pathological parameters and symptoms include, but are not limited to, fibrosis in the interstitium, inflammatory cell infiltration in the alveoli, shortness of breath, weight loss, fatigue, wheezing, chest pain and hemoptysis.

Some parameters of interstitial lung disease can be examined by non-invasive imaging procedures such as X rays, computerized tomography (CT) scan and/or magnetic resonance imaging (MRI), or by histological evaluation. For example, a histological examination of lung tissue can be conducted to assess the level of fibrosis in the interstitium. Bronchoscopy and bronchoalveolar lavage can be used to remove tissue or cells from the lower respiratory tract and to examine such cells for the presence of inflammatory infiltrates, e.g., leukocytes. Symptoms of interstitial lung disease are evaluated using standard means, such as spirometry, to assess lung function; blood tests to analyze O₂ and CO₂ content or O₂ saturation in the blood; peak flow monitoring to measure lung function; chest x-ray; computerized tomography scan; bronchoscopy; and the like.

In some embodiments, an “effective amount” of a TLR agonist is an amount effective to suppress TGF-β signaling in an epithelial cell of a lung. Whether TGF-β signaling is suppressed can be determined by measuring various parameters, e.g., an increase in MMP mRNA levels such as MMP-3, MMP-8, MMP-9, MMP-12 and MMP-13 and/or their enzymatic activities. Thus, in some embodiments, an effective amount of a TLR agonist is an amount that is effective to increase MMPs (e.g., MMP3, MMP8, MMP9, MMP12, and MMP13) mRNA and/or enzyme levels in lung tissue by at least about 10%, at least about 20%, at least about 25%, at least about 30%, at least about 40%, at least about 50%, at least about 60%, at least about 70%, at least about 80%, at least about 90%, at least about 2-fold, at least about 3-fold, at least about 4-fold, at least about 5-fold, or more, compared to the level of MMPs (e.g., MMP3, MMP8, MMP9, MMP12, MMP13) mRNA and/or enzyme in lung tissue from an individual not treated with the TLR agonist.

In some embodiments, an “effective amount” of a TLR agonist is an amount effective to increase the level of MMP mRNA and/or protein, including, e.g., MMP3 (stromelysin 1), MMP8 (neutrophil collagenase), MMP9 (gelatinase B), MMP12 (macrophage elastase), and MMP13 (collagenase 3). Thus, in some embodiments, an effective amount of a TLR agonist is an amount that is effective to increase a level of an MMP mRNA and/or enzyme levels in lung tissue by at least about 10%, at least about 20%, at least about 25%, at least about 30%, at least about 40%, at least about 50%, at least about 60%, at least about 70%, at least about 80%, at least about 90%, at least about 2-fold, at least about 3-fold, at least about 4-fold, at least about 5-fold, or more, compared to the level of the MMP mRNA and/or enzyme in lung tissue from an individual not treated with the TLR agonist.

In some embodiments, an effective amount of a TLR agonist is an amount that is effective to reduce integrin αᵥβ₆ mRNA and/or protein levels in lung tissue. In some embodiments, an effective amount of a TLR agonist is an amount that is effective to reduce integrin αᵥβ₆ mRNA and/or protein levels in lung tissue by at least about 10%, at least about 20%, at least about 25%, at least about 30%, at least about 40%, at least about 50%, at least about 60%, at least about 70%, at least about 80%, or at least about 90%, or more, compared to the level of integrin αᵥβ₆ mRNA and/or protein in lung tissue in an individual not treated with the TLR agonist.

In some embodiments, an “effective amount” of a TLR agonist is an amount effective to increase at least one parameter of lung function, e.g., an effective amount of a TLR agonist increases at least one parameter of lung function by at least about 10%, at least about 20%, at least about 25%, at least about 30%, at least about 40%, at least about 50%, at least about 60%, at least about 70%, at least about 80%, at least about 90%, at least about 2-fold, at least about 3-fold, at least about 4-fold, at least about 5-fold, or more, compared to an untreated individual or a placebo-treated control individual.

Parameters or measures of lung function include, but are not limited to, forced expiratory capacity in 1 second (FEV₁); forced vital capacity (FVC); diffusing capacity (DL_{co}; the lung diffusing capacity for carbon monoxide, expressed as mL CO/mm Hg/second); residual volume (RV); total lung capacity (TLC); lung compliance; V/Q (which describes ventilation/perfusion mismatch) and the like. Lung function can be measured using any known method, including, but not limited to spirometry, peak flow monitoring, and the like.

In some embodiments, an “effective amount” of a TLR agonist is an amount effective to increase the FVC by at least about 10% at least about 20%, at least about 25%, at least about 30%, at least about 40%, at least about 50%, at least about 60%, at least about 70%, at least about 80%, at least about 90%, at least about 2-fold, at least about 3-fold, at least about 4-fold, at least about 5-fold, or more compared to baseline or compared to placebo control.

In some of these embodiments, an “effective amount” of a TLR agonist is an amount that increases the single breath DL_{co} by at least about 15%, at least about 20%, at least about 30%, at least about 40%, at least about 50%, at least about 60%, at least about 70%, at least about 80%, at least about 90%, at least about 2-fold, at least about 3-fold, at least about 4-fold, at least about 5-fold, or more, compared to baseline. DL_{co} is the lung diffusing capacity for carbon monoxide, and is expressed as mL CO/mm Hg/second.

In some embodiments, an “effective amount” of a TLR agonist is an amount effective to increase progression-free survival period (arrests progression of the interstitial lung disease), e.g., the time from baseline (e.g., a time point from 1 day to about 30 days before beginning of treatment) to death or disease progression is increased by at least about 10%, at least about 20%, at least about 25%, at least about 30%, at least about 40%, at least about 50%, at least about 60%, at least about 70%, at least about 80%, at least about 90%, at least about 2-fold, at least about 3-fold, at least about 4-fold, at least about 5-fold, or more, compared a placebo-treated control individual. Thus, e.g., an effective amount of a TLR agonist is an amount effective to increase the progression-free survival time by at least about 1 week, at least about 2 weeks, at least about 3 weeks, at least about 4 weeks, at least about 2 months, at least about 3 months, at least about 4 months, at least about 5 months, at least about 6 months, at least about 8 months, at least about 10 months, at least about 12 months, at least about 18 months, at least about 2 years, at least about 3 years, or longer, compared to a placebo-treated control.

In other embodiments, an effective amount of a TLR agonist is an amount that slows progression of the interstitial lung disease. In these embodiments, an effective amount of a TLR agonist is an amount that is effective to slow progression of the interstitial lung disease by at least about 10%, at least about 15%, at least about 20%, or at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%, compared to the expected rate of progression in an individual having interstitial lung disease and not treated with the TLR agonist.

Disease progression in interstitial lung disease is the occurrence of one or more of the following: (1) a decrease in predicted FVC of 10% or more; (2) an increase in alveolar:arterial (A-a) gradient of 5 mm Hg or more; and (3) a decrease of 15% of more in single breath DL_{co}. Whether disease progression has occurred is determined by measuring one or more of these parameters on two consecutive occasions 2-20 weeks apart, e.g., 4 to 14 weeks apart, and comparing the value to baseline or to placebo control.

Thus, e.g., where an untreated or placebo-treated individual exhibits a 50% decrease in FVC over a period of time, an individual administered with an effective amount of a TLR agonist exhibits a decrease in FVC of 45%, about 42%, about 40%, about 37%, about 35%, about 32%, about 30%, or less, over the same time period.

In other embodiments, an effective amount of a TLR agonist is an amount that increases the probability of survival of the individual having interstitial lung disease, e.g., where the interstitial lung disease is a results of a disorder that is associated with a high mortality rate (e.g., idiopathic pulmonary fibrosis). For example, in these embodiments, an effective amount of a TLR agonist is an amount effective to increase the probability of survival of an individual having interstitial lung disease by at least about 10%, at least about 15%, at least about 20%, or at least about 25%, or more, compared to the expected probability of survival without administration of the TLR agonist.

In some embodiments, an effective amount of a TLR agonist is an amount that reduces the risk of death in an individual having interstitial lung disease, particularly where the fibrotic condition is associated with a high mortality rate. For example, the risk of death in an individual having interstitial lung disease and treated with a TLR agonist is reduced at least 2-fold, at least 2.5-fold, at least 3-fold, at least 3.5-fold, or at least 4-fold, or less, compared to the expected risk of death in an individual having interstitial lung disease and not treated with the TLR agonist.

Whether a given TLR agonist, alone or in combination therapy, as described below, is effective to treat interstitial lung disease can be readily determined. For example, a bleomycin-induced rodent model of lung fibrosis can be used to assess the efficacy of a therapeutic agent. Bleomycin-induced rodent models of lung fibrosis are amply described in the literature, e.g., in Giri et al. (1980) Exp. Mol. Pathol. 33:1-14; Thrall et al. (1979) Am. J. Pathol. 95:117-130; Zuckerman et al. (1980) J. Pharmacol. Exp. Ther. 213:425-431; and Iyer et al. (1995) J. Lab. Clin. Med. 125:779-785. For example, intratracheal instillation of bleomycin (7.5. units/kg/5 ml) in hamsters induces lung fibrosis. As another example, mice are given one dose of bleomycin (3.2 U/kg, intratracheal) twice daily for 14 days. Lung fibrosis can be assessed by measuring (1) lung hydroxyproline content as an index of collagen accumulation, (2) airway dysfunction by whole body plethysmography, and (3) histopathology. In a patient, efficacy of treatment with a TLR agonist is readily determined by, e.g., examination of a lung biopsy sample for interstitial fibrosis and/or by assessment of lung function, e.g., by spirometry.

TLR Agonists

A subject method involves administration of a therapeutically effective amount of a TLR ligand, generally a TLR agonist. A TLR agonist is any compound or substance that functions to activate a TLR, e.g., to induce a signaling event mediated by a TLR signal transduction pathway. An example of a TLR ligand-mediated signal transduction event is activation of the IL-1R-associated kinase, IRAK. Medzhitove et al. (1998) Mol. Cell 2:253-258; and Cao et al. (1996) Science 1128-1131. TLR include TLR1, TLR2, TLR3, TLR4, TLR5, TLR6, TLR7, TLR8, TLR9, and TLR10. Ozinsky et al. (2000) Proc. Natl. Acad. Sci. USA 97:13766-13771; and Akira and Hemmi (2003) Immunol. Lett. 85:85-95. TLR ligands include naturally-occurring TLR ligands, derivatives of naturally-occurring ligands, recombinant TLR ligands, and synthetic TLR ligands.

Suitable TLR agonists for use in a subject method include TLR agonists that reduce integrin αᵥβ₆ mRNA and/or protein levels in lung tissue. Whether a TLR agonist reduces integrin αᵥβ₆ mRNA and/or protein levels in lung tissue can be determined by detecting a level of integrin αᵥβ₆ mRNA and/or protein in lung tissue (e.g., a lung biopsy sample). Methods of detecting integrin αᵥβ₆ mRNA levels are well known in the art and include, but are not limited to, a polymerase chain reaction (PCR), e.g., reverse transcription-PCR (RT-PCR), quantitative PCR, quantitative RT-PCR, etc., with primers specific for integrin αᵥβ₆ mRNA; Northern blot analysis with probes specific for integrin αᵥβ₆ mRNA; and the like. Methods of detecting integrin αᵥβ₆ protein levels are well known in the art, and include, but are not limited to, immunological assays such as enzyme-linked immunosorbent assays (ELISA), protein blot (“Western blot”) assays, immunoprecipitation, and the like, where antibody specific for integrin αᵥβ₆ protein is used.

Suitable TLR agonists reduce integrin αᵥβ₆ mRNA and/or protein levels in lung tissue by at least about 10%, at least about 15%, at least about 20%, at least about 25%, at least about 30%, at least about 35%, at least about 40%, at least about 45%, at least about 50%, at least about 60%, at least about 70%, or at least about 80%, compared to the level of integrin αᵥβ₆ mRNA and/or protein in lung tissue in an untreated or placebo control individual.

TLR1 functions in signaling as a dimer with TLR2. TLR1 agonists include, but are not limited to, tri-acylated lipopeptides, phenol-soluble modulin, lipopeptide from Mycobacterium tuberculosis, OSP A lipopeptide from Borrelia burgdorferi; and the like.

TLR2 ligands include, but are not limited to, bacterial or synthetic lipopetides, lipoproteins (including naturally-occurring lipoproteins; derivatives of naturally-occurring lipoproteins; synthetic lipoproteins); lipopeptides (Takeuchi et al. (2000) J. Immunol. 164:554-557), e.g., lipopeptides from Mycobacteria tuberculosis, Borrelia burgdorferi, Treponema pallidum, etc.; whole bacteria, e.g., heat-killed Acholeplasma laidlawii, heat-killed Listeria monocytogenes (Flo et al. (2000) J. Immunol. 164:2064-2069), and the like; lipoteichoic acids (Schwandner et al. (1999) J. Biol. Chem. 274:17406-17409); peptidoglycans (Takeuchi et al. (1999) Immunity 11:443-451), e.g., peptidoglycans from Staphylococcus aureus, etc.; mannuronic acids; Neisseria porins; bacterial fimbriae, Yersinia virulence factors, cytomegalovirus virions, measles haemagglutinin; yeast cell wall extracts; yeast particle zymosan; glycosyl phosphatidyl inositol (GPI) anchor from Trypanosoma cruzi; and the like. An exemplary, non-limiting TLR2 ligand is Pam₃Cys (tripalmitoyl-S-glyceryl cysteine). Aliprantis et al. (1999) Science 285:736-739. Derivatives of Pam₃Cys are also suitable TLR2 agonists, where derivatives include, but are not limited to, Pam₃Cys-Ser-Ser-Asn-Ala; Pam₃Cys-Ser-(Lys)₄, and the like.

TLR3 ligands include naturally-occurring double-stranded RNA (dsRNA); synthetic ds RNA; and synthetic dsRNA analogs; and the like. Alexopoulou et al. (2001) Nature 413:732-738. An exemplary, non-limiting example of a synthetic ds RNA analog is poly(I:C).

TLR4 ligands include naturally-occurring lipopolysaccharides (LPS), e.g., LPS from a wide variety of Gram negative bacteria; derivatives of naturally-occurring LPS; synthetic LPS; bacteria heat shock protein-60 (Hsp60); mannuronic acid polymers; flavolipins; teichuronic acids; S. pneumoniae pneumolysin; bacterial fimbriae, respiratory syncytial virus coat protein; and the like.

TLR5 ligands include flagellin, e.g., naturally-occurring flagellin, recombinant flagellin, synthetic flagellin, flagellin fragments; and the like.

TLR 6 ligands include mycoplasma lipoproteins; lipoteichoic acid; bacterial peptidoglycans; di-acylated lipopeptides; peptidoglycan; phenol-soluble modulin; and the like.

TLR7 ligands include imidazoquinoline compounds; guanosine analogs; pyrimidinone compounds such as bropirimine and bropirimine analogs; and the like. Imidazoquinoline compounds that function as TLR7 ligands include, but are not limited to, imiquimod, (also known as Aldara, R-837, S-26308), and R-848 (also known as resiquimod, S-28463). Guanosine analogs that function as TLR7 ligands include certain C8-substitutes and N7,C8-disubstituted guanine ribonucleotides and deoxyribonucleotides, including, but not limited to, Loxoribine (7-allyl-8-oxoguanosine), 7-thia-8-oxo-guanosine (TOG), 7-deazaguanosine, and 7-deazadeoxyguanosine. Lee et al. (2003) Proc. Natl. Acad. Sci. USA 100:6646-6651. Bropirimine (PNU-54461), a 5-halo-6-phenyl-pyrimidinone, and bropirimine analogs are described in the literature and are also suitable for use. See, e.g., Vroegop et al. (1999) Intl. J. Immunopharmacol. 21:647-662.

TLR8 ligands include, but are not limited to, compounds such as R-848.

Examples of TLR9 ligands include nucleic acids comprising the sequence 5′-CG-3′, particularly where the C is unmethylated. Such TLR9 ligands are referred to as “therapeutic nucleic acids” herein and are discussed in detail below.

TLR 9 Agonists

As noted above, TLR9 agonists include nucleic acids comprising the sequence 5′-CG-3′, e.g., 5′-TCG-3′, particularly where the C is unmethylated. Such TLR9 ligands are referred to as “therapeutic nucleic acids” herein.

The terms “polynucleotide,” and “nucleic acid,” as used interchangeably herein in the context of therapeutic nucleic acid molecules, is a polynucleotide as defined above, and encompasses, inter alia, single- and double-stranded oligonucleotides (including deoxyribonucleotides, ribonucleotides, or both), modified oligonucleotides, and oligonucleosides, alone or as part of a larger nucleic acid construct, or as part of a conjugate with a non-nucleic acid molecule such as a polypeptide. Thus a therapeutic nucleic acid may be, for example, single-stranded DNA (ssDNA), double-stranded DNA (dsDNA), single-stranded RNA (ssRNA) or double-stranded RNA (dsRNA). Therapeutic nucleic acids also encompasses crude, detoxified bacterial (e.g., mycobacterial) RNA or DNA, as well as enriched plasmids enriched for a therapeutic nucleic acid. In some embodiments, a “therapeutic nucleic acid-enriched plasmid” refers to a linear or circular plasmid that comprises or is engineered to comprise a greater number of CpG motifs than normally found in mammalian DNA.

Exemplary, non-limiting therapeutic nucleic acid-enriched plasmids are described in, for example, Roman et al. (1997) Nat Med. 3(8):849-54. Modifications of oligonucleotides include, but are not limited to, modifications of the 3′OH or 5′OH group, modifications of the nucleotide base, modifications of the sugar component, and modifications of the phosphate group.

A therapeutic nucleic acid may comprise at least one nucleoside comprising an L-sugar. The L-sugar may be deoxyribose, ribose, pentose, deoxypentose, hexose, deoxyhexose, glucose, galactose, arabinose, xylose, lyxose, or a sugar “analog” cyclopentyl group. The L-sugar may be in pyranosyl or furanosyl form.

Therapeutic nucleic acids generally do not provide for nor is there any requirement that they provide for, expression of any amino acid sequence encoded by the polynucleotide, and thus the sequence of a therapeutic nucleic acid may be, and generally is, non-coding. Therapeutic nucleic acids may comprise a linear double or single-stranded molecule, a circular molecule, or can comprise both linear and circular segments. Therapeutic nucleic acids may be single-stranded, or may be completely or partially double-stranded.

In some embodiments, a therapeutic nucleic acid for use in a subject method is an oligonucleotide, e.g., consists of a sequence of from about 5 to about 200, from about 10 to about 100, from about 12 to about 50, from about 15 to about 25, from about 5 to about 15, from about 5 to about 10, or from about 5 to about 7 nucleotides in length. In some embodiments, a therapeutic nucleic acid that is less than about 15, less than about 12, less than about 10, or less than about 8 nucleotides in length is associated with a larger molecule, e.g., adsorbed onto an insoluble support, as described below.

In some embodiments, a therapeutic nucleic acid does not provide for expression of a peptide or polypeptide in a eukaryotic cell, e.g., introduction of a therapeutic nucleic acid into a eukaryotic cell does not result in production of a peptide or polypeptide, because the therapeutic nucleic acid does not provide for transcription of an mRNA encoding a peptide or polypeptide. In these embodiments, a therapeutic nucleic acid lacks promoter regions and other control elements necessary for transcription in a eukaryotic cell.

A therapeutic nucleic acid can be isolated from a bacterium, e.g., separated from a bacterial source; synthetic (e.g., produced by standard methods for chemical synthesis of polynucleotides); produced by standard recombinant methods, then isolated from a bacterial source; or a combination of the foregoing. In many embodiments, a therapeutic nucleic acid is purified, e.g., is at least about 80%, at least about 90%, at least about 95%, at least about 98%, at least about 99%, or more, pure.

In other embodiments, a therapeutic nucleic acid is part of a larger nucleotide construct (e.g., a plasmid vector, a viral vector, or other such construct). A wide variety of plasmid and viral vector are known in the art, and need not be elaborated upon here. A large number of such vectors has been described in various publications, including, e.g., Current Protocols in Molecular Biology, (F. M. Ausubel, et al., Eds. 1987, and updates). Many vectors are commercially available.

Therapeutic Nucleic Acids Comprising a CpG Motif

In general, a therapeutic nucleic acid used in a subject method comprise at least one unmethylated CpG motif. The relative position of any CpG sequence in a polynucleotide in certain mammalian species (e.g., rodents) is 5′-CG-3′ (i.e., the C is in the 5′ position with respect to the G in the 3′ position).

In some embodiments, a therapeutic nucleic acid comprises a central palindromic core sequence comprising at least one CpG sequence, where the central palindromic core sequence contains a phosphodiester backbone, and where the central palindromic core sequence is flanked on one or both sides by phosphorothioate backbone-containing polyguanosine sequences.

In other embodiments, a therapeutic nucleic acid comprises one or more TCG sequences at or near the 5′ end of the nucleic acid; and at least two additional CG dinucleotides. In some of these embodiments, the at least two additional CG dinucleotides are spaced three nucleotides, two nucleotides, or one nucleotide apart. In some of these embodiments, the at least two additional CG dinucleotides are contiguous with one another. In some of these embodiments, the therapeutic nucleic acid comprises (TCG)n, where n=one to three, at the 5′ end of the nucleic acid. In other embodiments, the therapeutic nucleic acid comprises (TCG)n, where n=one to three, and where the (TCG)n sequence is flanked by one nucleotide, two nucleotides, three nucleotides, four nucleotides, or five nucleotides, on the 5′ end of the (TCG)n sequence.

Exemplary consensus CpG motifs of therapeutic nucleic acids useful in the invention include, but are not necessarily limited to:

5′-Purine-Purine-(C)-(G)-Pyrimidine-Pyrimidine-3′, in which the therapeutic nucleic acid comprises a CpG motif flanked by at least two purine nucleotides (e.g., GG, GA, AG, AA, II, etc.,) and at least two pyrimidine nucleotides (CC, TT, CT, TC, UU, etc.);

5′-Purine-TCG-Pyrimidine-Pyrimidine-3′;

5′-TCG-N-N-3′; where n is any base;

5′-(TCG)ₙ-3′, where n is any integer that is 1 or greater, e.g., to provide a TCG-based therapeutic nucleic acid (e.g., where n=3, the polynucleotide comprises the sequence 5′-TCGNNTCGNNTCG-3′);

5′ Nₘ-(TCG)n-Nₚ-3′, where N is any nucleotide, where m is zero, one, two, or three, where n is any integer that is 1 or greater, and where p is one, two, three, or four;

5′ Nₘ-(TCG)ₙ-Nₚ-3′, where N is any nucleotide, where m is zero to 5, and where n is any integer that is 1 or greater, where p is four or greater, and where the sequence N-N-N-N comprises at least two CG dinucleotides that are either contiguous with each other or are separated by one nucleotide, two nucleotides, or three nucleotides; and

5′-Purine-Purine —CG-Pyrimidine-TCG-3′.

A non-limiting example of a nucleic acid comprising 5′-(TCG)ₙ-3′, where n is any integer that is 1 or greater, is a nucleic acid comprising the sequence

| | |
|---|---|
| 5′ TCGTCGTTTTGTCGTTTTGTCGTT 3′. | (SEQ ID NO:05) |

Where a nucleic acid comprises a sequence of the formula: 5′-Nₘ-(TCG)ₙ-Nₚ-3′, where N is any nucleotide, where m is zero to 5, and where n is any integer that is 1 or greater, where p is four or greater, and where the sequence N-N-N-N comprises at least two CG dinucleotides that are either contiguous with each other or are separated by one nucleotide, two nucleotides, or three nucleotides, exemplary therapeutic nucleic acids useful in the invention include, but are not necessarily limited to:

| | |
|---|---|
| (1) | a sequence of the formula in which n = 2, |
| | and N^{P} is NNCGNNCG; |
| (2) | a sequence of the formula in which n = 2, |
| | and N^{P} is AACGTTCG; |
| (3) | a sequence of the formula in which n = 2, |
| | and N^{P} is TTCGAACG; |
| (4) | a sequence of the formula in which n = 2, |
| | and N^{P} is TACGTACG; |
| (5) | a sequence of the formula in which n = 2, |
| | and N^{P} is ATCGATCG; |
| (6) | a sequence of the formula in which n = 2, |
| | and N^{P} is CGCGCGCG; |
| (7) | a sequence of the formula in which n = 2, |
| | and N^{P} is GCCGGCCG; |
| (8) | a sequence of the formula in which n = 2, |
| | and N^{P} is CCCGGGCG; |
| (9) | a sequence of the formula in which n = 2, |
| | and N^{P} is GGCGCCCG; |
| (10) | a sequence of the formula in which n = 2, |
| | and N^{P} is CCCGTTCG; |
| (11) | a sequence of the formula in which n = 2, |
| | and N^{P} is GGCGTTCG; |
| (12) | a sequence of the formula in which n = 2, |
| | and N^{P} is TTCGCCCG; |
| (13) | a sequence of the formula in which n = 2, |
| | and N^{P} is TTCGGGCG; |
| (14) | a sequence of the formula in which n = 2, |
| | and N^{P} is AACGCCCG; |
| (15) | a sequence of the formula in which n = 2, |
| | and N^{P} is AACGGGCG; |
| (16) | a sequence of the formula in which n = 2, |
| | and N^{P} is CCCGAACG; |
| | and |
| (17) | a sequence of the formula in which n = 2, |
| | and N^{P} is GGCGAACG; |

and where, in any of 1-17, m = zero, one,
two, or three.

Where a nucleic acid comprises a sequence of the formula: 5′ Nₘ-(TCG)n-Nₚ-3′, where N is any nucleotide, where m is zero, one, two, or three, where n is any integer that is 1 or greater, and where p is one, two, three, or four, exemplary therapeutic nucleic acids useful in the invention include, but are not necessarily limited to:

(1) a sequence of the formula where m=zero, n=1, and Nₚ is T-T-T;

(2) a sequence of the formula where m=zero, n=1, and Nₚ is T-T-T-T;

(3) a sequence of the formula where m=zero, n=1, and Nₚ is C-C-C-C;

(4) a sequence of the formula where m=zero, n=1, and Nₚ is A-A-A-A;

(5) a sequence of the formula where m=zero, n=1, and Nₚ is A-G-A-T;

(6) a sequence of the formula where Nₘ is T, n=1, and Nₚ is T-T-T;

(7) a sequence of the formula where Nₘ is A, n=1, and Nₚ is T-T-T;

(8) a sequence of the formula where Nₘ is C, n=1, and Nₚ is T-T-T;

(9) a sequence of the formula where Nₘ is G, n=1, and Nₚ is T-T-T;

(10) a sequence of the formula where Nₘ is T, n=1, and Nₚ is A-T-T;

(11) a sequence of the formula where Nₘ is A, n=1, and Nₚ is A-T-T; and

(12) a sequence of the formula where Nₘ is C, n=1, and Nₚ is A-T-T.

The core structure of a therapeutic nucleic acid useful in the invention may be flanked upstream and/or downstream by any number or composition of nucleotides or nucleosides. In some embodiments, the core sequence of a therapeutic nucleic acid is at least 6 bases or 8 bases in length, and the complete therapeutic nucleic acid (core sequences plus flanking sequences 5′, 3′ or both) is usually between 6 bases or 8 bases, and up to about 200 bases in length.

Exemplary DNA-based therapeutic nucleic acids useful in the invention include, but are not necessarily limited to, polynucleotides comprising one or more of the following nucleotide sequences:

AGCGCT, AGCGCC, AGCGTT, AGCGTC, AACGCT, AACGCC,
AACGTT, AACGTC, GGCGCT, GGCGCC, GGCGTT, GGCGTC,
GACGCT, GACGCC, GACGTT, GACGTC, GTCGTC, GTCGCT,
GTCGTT, GTCGCC, ATCGTC, ATCGCT, ATCGTT, ATCGCC,
TCGTCG, and TCGTCGTCG.

Exemplary DNA-based therapeutic nucleic acids useful in the invention include, but are not necessarily limited to, polynucleotides comprising the following octameric nucleotide sequences:

AGCGCTCG, AGCGCCCG, AGCGTTCG, AGCGTCCG, AACGCTCG,
AACGCCCG, AACGTTCG, AACGTCCG, GGCGCTCG, GGCGCCCG,
GGCGTTCG, GGCGTCCG, GACGCTCG, GACGCCCG, GACGTTCG,
and GACGTCCG.

A therapeutic nucleic acid useful in carrying out a subject method can comprise one or more of any of the above CpG motifs. For example, a therapeutic nucleic acid useful in the invention can comprise a single instance or multiple instances (e.g., 2, 3, 5 or more) of the same CpG motif. Alternatively, a therapeutic nucleic acid can comprise multiple CpG motifs (e.g., 2, 3, 5 or more) where at least two of the multiple CpG motifs have different consensus sequences, or where all CpG motifs in the therapeutic nucleic acid have different consensus sequences.

A therapeutic nucleic acid useful in the invention may or may not include palindromic regions. If present, a palindrome may extend only to a CpG motif, if present, in the cote hexamer or octamer sequence, or may encompass more of the hexamer or octamer sequence as well as flanking nucleotide sequences.

A therapeutic CpG-containing nucleic acid suitable for use in a subject method can be readily identified, e.g., by using an animal model of chronic asthma as described in the Examples, or using a bleomycin-induced animal model of lung fibrosis. A suitable nucleic acid, when administered in an effective amount, reduces at least one pathological parameter associated with airway remodeling by at least 10%, at least 15%, at least 20%, or at least 25% or more, when compared to a suitable control. Parameters associated with airway remodeling include mucus hypersecretion in the lungs; peribronchial trichrome staining; peribronchial collagen production; smooth muscle layer thickness; peribronchial myoblast α-smooth muscle actin production; and smooth muscle hypertrophy, hyperplasia, and differentiation into myoblasts. A suitable nucleic acid, when administered in an effective amount, reduces at least one pathological parameter associated with lung fibrosis by at least 10%, at least 15%, at least 20%, or at least 25% or more, when compared to a suitable control.

Modifications

A therapeutic nucleic acid suitable for use in a subject method can be modified in a variety of ways. For example, a therapeutic nucleic acid can comprise backbone phosphate group modifications (e.g., methylphosphonate, phosphorothioate, phosphoroamidate and phosphorodithioate internucleotide linkages), which modifications can, for example, enhance their stability in vivo, making them particularly useful in therapeutic applications. A particularly useful phosphate group modification is the conversion to the phosphorothioate or phosphorodithioate forms of a therapeutic nucleic acid. Phosphorothioates and phosphorodithioates are more resistant to degradation in vivo than their unmodified oligonucleotide counterparts, increasing the half-lives of the therapeutic nucleic acids and making them more available to the subject being treated.

Other modified therapeutic nucleic acids encompassed by the present invention include therapeutic nucleic acids having modifications at the 5′ end, the 3′ end, or both the 5′ and 3′ ends. For example, the 5′ and/or 3′ end can be covalently or non-covalently associated with a molecule (either nucleic acid, non-nucleic acid, or both) to, for example, increase the bio-availability of the therapeutic nucleic acid, increase the efficiency of uptake where desirable, facilitate delivery to cells of interest, and the like. Exemplary molecules for conjugation to a therapeutic nucleic acid include, but are not necessarily limited to, cholesterol, phospholipids, fatty acids, sterols, oligosaccharides, polypeptides (e.g., immunoglobulins), peptides, antigens (e.g., peptides, small molecules, etc.), linear or circular nucleic acid molecules (e.g., a plasmid), insoluble supports, and the like.

A therapeutic nucleic acid is in some embodiments linked (e.g., conjugated, covalently linked, non-covalently associated with, or adsorbed onto) an insoluble support. An exemplary, non-limiting example of an insoluble support is cationic poly(D,L-lactide-co-glycolide).

Additional therapeutic nucleic acid conjugates, and methods for making same, are known in the art and described in, for example, WO 98/16427 and WO 98/55495. Thus, the term “therapeutic nucleic acid” includes conjugates comprising a therapeutic nucleic acid.

A polypeptide, e.g., a therapeutic polypeptide, may be conjugated directly or indirectly, e.g., via a linker molecule, to a therapeutic nucleic acid. A wide variety of linker molecules are known in the art and can be used in the conjugates. The linkage from the peptide to the oligonucleotide may be through a peptide reactive side chain, or the N- or C-terminus of the peptide. Linkage from the oligonucleotide to the peptide may be at either the 3′ or 5′ terminus, or internal. A linker may be an organic, inorganic, or semi-organic molecule, and may be a polymer of an organic molecule, an inorganic molecule, or a co-polymer comprising both inorganic and organic molecules.

If present, the linker molecules are generally of sufficient length to permit oligonucleotides and/or polynucleotides and a linked polypeptide to allow some flexible movement between the oligonucleotide and the polypeptide. The linker molecules are generally about 6-50 atoms long. The linker molecules may also be, for example, aryl acetylene, ethylene glycol oligomers containing 2-10 monomer units, diamines, diacids, amino acids, or combinations thereof. Other linker molecules which can bind to oligonucleotides may be used in light of this disclosure.

Peptides may be synthesized chemically or enzymatically, may be produced recombinantly, may be isolated from a natural source, or a combination of the foregoing. Peptides may be isolated from natural sources using standard methods of protein purification known in the art, including, but not limited to, HPLC, exclusion chromatography, gel electrophoresis, affinity chromatography, or other purification technique. One may employ solid phase peptide synthesis techniques, where such techniques are known to those of skill in the art. See Jones, The Chemical Synthesis of Peptides (Clarendon Press, Oxford)(1994). Generally, in such methods a peptide is produced through the sequential additional of activated monomeric units to a solid phase bound growing peptide chain. Well-established recombinant DNA techniques can be employed for production of peptides.

Formulations, Dosages, and Routes of Administration

Formulations

In general, a TLR agonist is prepared in a pharmaceutically acceptable composition for delivery to a host. Pharmaceutically acceptable carriers suitable for use with a TLR agonist include sterile aqueous of non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, and microparticles, including saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers (such as those based on Ringer's dextrose), and the like. A composition comprising a TLR agonist may also be lyophilized using means well known in the art, for subsequent reconstitution and use according to the invention.

In general, the pharmaceutical compositions can be prepared in various forms, such as granules, tablets, pills, suppositories, capsules, suspensions, salves, lotions and the like. Pharmaceutical grade organic or inorganic carriers and/or diluents suitable for oral and topical use can be used to make up compositions comprising the therapeutically-active compounds. Diluents known to the art include aqueous media, vegetable and animal oils and fats. Stabilizing agents, wetting and emulsifying agents, salts for varying the osmotic pressure or buffers for securing an adequate pH value, and skin penetration enhancers can be used as auxiliary agents. Preservatives and other additives may also be present such as, for example, antimicrobials, antioxidants, chelating agents, and inert gases and the like.

A TLR agonist can be administered in the absence of agents or compounds that might facilitate uptake by target cells. A TLR agonist can be administered with compounds that facilitate uptake of such an agonist by target cells (e.g., by macrophages, bronchial smooth muscle cells, airway epithelial cells, etc.) or otherwise enhance transport of a TLR agonist to a treatment site for action.

Absorption promoters, detergents and chemical irritants (e.g., keratinolytic agents) can enhance transmission of TLR agonist composition into a target tissue (e.g., through the skin). For general principles regarding absorption promoters and detergents which have been used with success in mucosal delivery of organic and peptide-based drugs, see, e.g., Chien, Novel Drug Delivery Systems, Ch. 4 (Marcel Dekker, 1992). Examples of suitable nasal absorption promoters in particular are set forth at Chien, supra at Ch. 5, Tables 2 and 3; milder agents are preferred. Suitable agents for use in the method of this invention for mucosal/nasal delivery are also described in Chang, et al., Nasal Drug Delivery, “Treatise on Controlled Drug Delivery”, Ch. 9 and Tables 3-4B thereof, (Marcel Dekker, 1992). Suitable agents which are known to enhance absorption of drugs through skin are described in Sloan, Use of Solubility Parameters from Regular Solution Theory to Describe Partitioning-Driven Processes, Ch. 5, “Prodrugs: Topical and Ocular Drug Delivery” (Marcel Dekker, 1992), and at places elsewhere in the text. All of these references are incorporated herein for the sole purpose of illustrating the level of knowledge and skill in the art concerning drug delivery techniques.

A colloidal dispersion system may be used for targeted delivery of TLR agonist to specific tissue. Colloidal dispersion systems include macromolecule complexes, nanocapsules, microspheres, beads, and lipid-based systems including oil-in-water emulsions, micelles, mixed micelles, and liposomes:

Liposomes are artificial membrane vesicles which are useful as delivery vehicles in vitro and in vivo. It has been shown that large unilamellar vesicles (LUV), which range in size from 0.2-4.0 Fm can encapsulate a substantial percentage of an aqueous buffer comprising large macromolecules. RNA and DNA can be encapsulated within the aqueous interior and be delivered to cells in a biologically active form (Fraley, et al., (1981) Trends Biochem. Sci., 6:77). The composition of the liposome is usually a combination of phospholipids, particularly high-phase-transition-temperature phospholipids, usually in combination with steroids, especially cholesterol. Other phospholipids or other lipids may also be used. The physical characteristics of liposomes depend on pH, ionic strength, and the presence of divalent cations. Examples of lipids useful in liposome production include phosphatidyl compounds, such as phosphatidylglycerol, phosphatidylcholine, phosphatidylserine, phosphatidylethanolamine, sphingolipids, cerebrosides, and gangliosides. Particularly useful are diacylphosphatidylglycerols, where the lipid moiety contains from 14-18 carbon atoms, particularly from 16-18 carbon atoms, and is saturated. Illustrative phospholipids include egg phosphatidylcholine, dipalmitoylphosphatidylcholine and distearoylphosphatidylcholine. Exemplary liposome compositions suitable for use in a subject method are described in Louria-Hayon et al. (2002) Vaccine 20:3342.

Where desired, targeting of liposomes can be classified based on anatomical and mechanistic factors. Anatomical classification is based on the level of selectivity, for example, organ-specific, cell-specific, and organelle-specific. Mechanistic targeting can be distinguished based upon whether it is passive or active. Passive targeting utilizes the natural tendency of liposomes to distribute to cells of the reticulo-endothelial system (RES) in organs which contain sinusoidal capillaries. Active targeting, on the other hand, involves alteration of the liposome by coupling the liposome to a specific ligand such as a monoclonal antibody, sugar, glycolipid, or protein, or by changing the composition or size of the liposome in order to achieve targeting to organs and cell types other than the naturally occurring sites of localization.

The surface of the targeted delivery system may be modified in a variety of ways. In the case of a liposomal targeted delivery system, lipid groups can be incorporated into the lipid bilayer of the liposome in order to maintain the targeting ligand in stable association with the liposomal bilayer. Various well known linking groups can be used for joining the lipid chains to the targeting ligand (see, e.g., Yanagawa, et al., (1988) Nuc. Acids Symp. Ser., 19:189; Grabarek, et al., (1990) Anal. Biochem., 185:131; Staros, et al., (1986) Anal. Biochem. 156:220 and Boujrad, et al., (1993) Proc. Natl. Acad. Sci. USA, 90:5728). Targeted delivery of a TLR agonist can also be achieved by conjugation of the TLR agonist to a the surface of viral and non-viral recombinant expression vectors, to an antigen or other ligand, to a monoclonal antibody or to any molecule which has the desired binding specificity.

Routes of Administration

A TLR agonist is administered to an individual using any available method and route suitable for drug delivery, including in vivo and ex vivo methods, as well as systemic, mucosal, and localized routes of administration.

Conventional and pharmaceutically acceptable routes of administration include inhalational routes, intranasal, intramuscular, intratracheal, subcutaneous, intradermal, topical application, intravenous, rectal, nasal, oral and other enteral and parenteral routes of administration. Routes of administration may be combined, if desired, or adjusted depending upon the TLR agonist and/or the desired effect on the airway fibrotic disorder. The TLR agonist composition can be administered in a single dose or in multiple doses, and may encompass administration of booster doses, to elicit and/or maintain the desired effect on fibrosis, lung function, etc.

A TLR agonist can be administered to a host using any available conventional methods and routes suitable for delivery of conventional drugs, including systemic or localized routes. In general, routes of administration contemplated by the invention include, but are not necessarily limited to, enteral, parenteral, or inhalational routes. In some embodiments, administration is to the respiratory tract. Inhalational routes may be suitable for treatment of airway fibrosis.

The route of administration depends, in part, on the severity of the disease. Inhalational routes of administration (e.g., intranasal, intrapulmonary, and the like) may be particularly useful for treating fibrosis in the lung. Such means include inhalation of aerosol suspensions or insufflation of a TLR agonist composition. Nebulizer devices, metered dose inhalers, and the like suitable for delivery of polynucleotide compositions to the nasal mucosa, trachea and bronchioli are well-known in the art and will therefore not be described in detail here. For general review in regard to intranasal drug delivery, see, e.g., Chien, Novel Drug Delivery Systems, Ch. 5 (Marcel Dekker, 1992).

Parenteral routes of administration other than inhalation administration include, but are not necessarily limited to, topical, transdermal, subcutaneous, intramuscular, intraorbital, intraspinal, intrasternal, and intravenous routes, i.e., any route of administration other than through the alimentary canal. Parenteral administration can be carried to effect systemic or local delivery of a TLR agonist.

Systemic administration typically involves intravenous, intradermal, subcutaneous, or intramuscular administration or systemically absorbed topical or mucosal administration of pharmaceutical preparations. Mucosal administration includes administration to the respiratory tissue, e.g., by inhalation, nasal drops, and the like.

A TLR agonist can also be delivered to the subject by enteral administration. Enteral routes of administration include, but are not necessarily limited to, oral and rectal (e.g., using a suppository) delivery.

Methods of administration of a TLR agonist through the skin or mucosa include, but are not necessarily limited to, topical application of a suitable pharmaceutical preparation, transdermal transmission, injection and epidermal administration. For transdermal transmission, absorption promoters or iontophoresis are suitable methods. For review regarding such methods, those of ordinary skill in the art may wish to consult Chien, supra at Ch. 7. Iontophoretic transmission may be accomplished using commercially available “patches” which deliver their product continuously via electric pulses through unbroken skin for periods of several days or more. An exemplary patch product for use in this method is the LECTRO PATCH™ (manufactured by General Medical Company, Los Angeles, Calif.) which electronically maintains reservoir electrodes at neutral pH and can be adapted to provide dosages of differing concentrations, to dose continuously and/or to dose periodically.

Formulations Suitable for Inhalation

Delivery of a TLR agonist is, in some embodiments, via insufflation of an flowable formulation comprising the TLR agonist, where the flowable formulation is one that is suitable for delivery by inhalation, e.g., an aerosolized formulation. The present invention thus provides compositions comprising a TLR agonist and a formulation suitable for delivery by inhalation, e.g., an aerosolized formulation or other flowable formulation suitable for delivery by inhalation. As used herein, the term “aerosol” is used in its conventional sense as referring to very fine liquid or solid particles carries by a propellant gas under pressure to a site of therapeutic application. The term “liquid formulation for delivery to respiratory tissue” and the like, as used herein, describe compositions comprising a TLR agonist with a pharmaceutically acceptable carrier in flowable liquid form. Such formulations, when used for delivery to a respiratory tissue, are generally solutions, e.g. aqueous solutions, ethanolic solutions, aqueous/ethanolic solutions, saline solutions and colloidal suspensions.

In general, aerosolized particles for respiratory delivery must have a diameter of 12 microns or less. Typically, the particle size varies with the site targeted (e.g, delivery targeted to the bronchi, bronchia, bronchioles, alveoli, or circulatory system). For example, topical lung treatment can be accomplished with particles having a diameter in the range of 1.0 to 12.0 microns. Effective systemic treatment requires particles having a smaller diameter, generally in the range of 0.5 to 6.0 microns. Thus, in some embodiments, at least about 40%, at least about 50%, at least about 60%, at least about 70%, at least about 80%, at least about 90%, or more, of an aerosolized formulation comprising a TLR agonist for delivery to a respiratory tissue is composed of particles in the range of from about 0.5 to about 12 micrometers, from about 0.5 to about 6 micrometers, or from about 1.0 to about 12 micrometers.

The formulation for delivery to a respiratory tissue may be provided in a container suitable for delivery of aerosolized formulations. Thus, the present invention provides a container suitable for delivery of an aerosolized formulation, the container comprising a subject formulation comprising a TLR agonist and a formulation suitable for delivery by inhalation. U.S. Pat. Nos. 5,544,646; 5,709,202; 5,497,763; 5,544,646; 5,718,222; 5,660,166; 5,823,178; 5,829,435; and 5,906,202 describe devices and methods useful in the generation of aerosols suitable for drug delivery, any of which can be used in the present invention for delivering a formulation comprising a TLR agonist to a respiratory tissue.

In some embodiments, the invention provides a container, which may be a disposable container, having at least one wall that is collapsible or movable upon application of a force, wherein at least one wall has an opening. A porous membrane having pores in a range of from about 0.25 microns (micrometers) to about 6 microns covers the opening. The container comprises a flowable liquid formulation comprising a TLR agonist. Upon application of a force, the flowable liquid formulation is forced through the pores in the membrane and is aerosolized. The container may, be provided in any known configuration, e.g., a blister pack. The container may be provided together with an aerosol delivery device, such that the aerosolized formulation exits the container and proceeds through a channel in an aerosol delivery device and into the respiratory tract of an individual.

When a pharmaceutical aerosol is employed in this invention, the aerosol contains a TLR agonist, which can be dissolved, suspended, or emulsified in a mixture of a fluid carrier and a propellant. The aerosol can be in the form of a solution, suspension, emulsion, powder, or semi-solid preparation. Aerosols employed in the present invention are intended for administration as fine, solid particles or as liquid mists via the respiratory tract of a patient. Various types of propellants known to one of skill in the art can be utilized. Examples of suitable propellants include, but is not limited to, hydrocarbons or other suitable gas. In the case of the pressurized aerosol, the dosage unit may be determined by providing a value to deliver a metered amount.

Administration of formulation comprising a TLR agonist can also be carried out with a nebulizer, which is an instrument that generates very fine liquid particles of substantially uniform size in a gas. For example, a liquid containing a TLR agonist is dispersed as droplets. The small droplets can be carried by a current of air through an outlet tube of the nebulizer. The resulting mist penetrates into the respiratory tract of the patient.

A powder composition containing a TLR agonist, with or without a lubricant, carrier, or propellant, can be administered to a mammal in need of therapy. This embodiment of the invention can be carried out with a conventional device for administering a powder pharmaceutical composition by inhalation. For example, a powder mixture of the compound and a suitable powder base such as lactose or starch may be presented in unit dosage form in for example capsular or cartridges, e.g. gelatin, or blister packs, from which the powder may be administered with the aid of an inhaler.

Combination therapies may be used to treat a respiratory condition (e.g., to increase lung function), as described herein. In particular, a TLR agonist may be combined with conventional therapeutic agents for treating various respiratory diseases such as asthma, bronchitis, etc.

The present invention is intended to encompass the free acids, free bases, salts, amines and various hydrate forms including semi-hydrate forms of such respiratory drugs and is particularly directed towards pharmaceutically acceptable formulations of such drugs which are formulated in combination with pharmaceutically acceptable excipient materials generally known to those skilled in the art—in some embodiments without other additives such as preservatives. In some embodiments, drug formulations do not include additional components which have a significant effect on the overall formulation such as preservatives. Thus certain formulations consist essentially of pharmaceutically active drug and a pharmaceutically acceptable carrier (e.g., water and/or ethanol). However, if a drug is liquid without an excipient the formulation may consist essentially of the drug which has a sufficiently low viscosity that it can be aerosolized using a dispenser.

Administration by inhalation will be carried out in some embodiments of the invention, because smaller doses can be delivered locally to the specific cells (e.g., cells of respiratory tissue, bronchial smooth muscle cells, airway epithelial cells, airway macrophages, etc.) which are most in need of treatment. By delivering smaller doses, any adverse side effects are eliminated or substantially reduced. By delivering directly to the cells which are most in need of treatment, the effect of the treatment will be realized more quickly.

There are several different types of inhalation methodologies which can be employed in connection with the present invention. A TLR agonist can be formulated in basically three different types of formulations for inhalation. First, a TLR agonist can be formulated with low boiling point propellants. Such formulations are generally administered by conventional meter dose inhalers (MDI's). However, conventional MDI's can be modified so as to increase the ability to obtain repeatable dosing by utilizing technology which measures the inspiratory volume and flow rate of the patient as discussed within U.S. Pat. Nos. 5,404,871 and 5,542,410.

Alternatively, a TLR agonist can be formulated in aqueous or ethanolic solutions and delivered by conventional nebulizers. In many instances, such solution formulations are aerosolized using devices and systems such as disclosed within U.S. Pat. Nos. 5,497,763; 5,544,646; 5,718,222; and 5,660,166.

In addition, a TLR agonist can be formulated into dry powder formulations. Such formulations can be administered by simply inhaling the dry powder formulation after creating an aerosol mist of the powder. Technology for carrying such out is described within U.S. Pat. No. 5,775,320 and U.S. Pat. No. 5,740,794.

With respect to each of the patents recited above, applicants point out that these patents cite other publications in intrapulmonary drug delivery and such publications can be referred to for specific methodology, devices and formulations which could be used in connection with the delivery of a TLR agonist. Further, each of the patents are incorporated herein by reference in their entirety for purposes of disclosing formulations, devices, packaging and methodology for the delivery of TLR agonist formulations.

Dosages

Although the dosage used will vary depending on the clinical goals to be achieved, a suitable dose range is one which provides up to about 1 μg to about 1,000 μg, from about 1,000 μg to about 10,000 μg, or from about 10 mg to about 100 mg of a TLR agonist can be administered in a single dosage. Alternatively, a target dose of a TLR agonist can be considered to be about 1-1.0 μM in a sample of host blood drawn within the first 24-48 hours after administration of a TLR agonist.

The therapeutic activity of a TLR agonist is generally dose-dependent. Therefore, to increase a TLR agonist's potency by a magnitude of two, each single dose is doubled in concentration. Increased dosages may be needed to achieve the desired therapeutic goal. The invention thus contemplates administration of multiple doses.

In many embodiments, multiple doses of a TLR agonist are administered. For example, a TLR agonist is administered once per month, twice per month, three times per month, every other week (qow), once per week (qw), twice per week (biw), three times per week (tiw), four times per week, five times per week, six times per week, every other day (qod), daily (qd), twice a day (bid), or three times a day (tid), substantially continuously, or continuously, over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more.

Combination Therapies

In some embodiments, two or more TLR agonists are administered in combination therapy. In other embodiments, a TLR agonist is administered in combination therapy with one or more additional therapeutic agents.

Combination Therapy with Two or More TLR Agonists

In some embodiments, two or more TLR agonists are administered in combination therapy. In some embodiments, a subject combination therapy involves administering an effective amount of a TLR9 agonist and an effective amount of a TLR2 agonist. In some embodiments, a subject combination therapy involves administering an effective amount of a TLR9 agonist and an effective amount of a TLR7 agonist. In some embodiments, a subject combination therapy involves administering an effective amount of a TLR9 agonist and an effective amount of a TLR3 agonist. In some embodiments, a subject combination therapy involves administering an effective amount of a TLR9 agonist and an effective amount of a TLR8 agonist.

Combination Therapy with an Additional Therapeutic Agent

In some embodiments, a TLR agonist is administered in combination therapy with one or more additional therapeutic agents. The choice of the additional therapeutic agent will depend, in part, on the specific condition being treated.

A TLR agonist will in some embodiments be administered to an individual in combination (e.g., in the same formulation or in separate formulations) with another therapeutic agent (“combination therapy”). The TLR agonist can be administered in admixture with another therapeutic agent or can be administered in a separate formulation. When administered in separate formulations, a TLR agonist and another therapeutic agent can be administered substantially simultaneously (e.g., within about 60 minutes, about 50 minutes, about 40 minutes; about 30 minutes, about 20 minutes, about 10 minutes, about 5 minutes, or about 1 minute of each other) or separated in time by about 1 hour, about 2 hours, about 4 hours, about 6 hours, about 10 hours, about 12 hours, about 24 hours, about 36 hours, or about 72 hours, or more.

Therapeutic agents for treating respiratory diseases which may be administered in combination with a TLR agonist in a subject method include, but are not limited to beta adrenergics which include bronchodilators including albuterol, isoproterenol sulfate, metaproterenol sulfate, terbutaline sulfate, pirbuterol acetate and salmeterol formotorol; steroids including beclomethasone dipropionate, flunisolide, fluticasone, budesonide and triamcinolone acetonide. Anti-inflammatory drugs used in connection with the treatment of respiratory diseases include steroids such as beclomethasone dipropionate, triamcinolone acetonide, flunisolide and fluticasone. Other anti-inflammatory drugs include cromoglycates such as cromolyn sodium. Other respiratory drugs which would qualify as bronchodilators include anticholenergics including ipratropium bromide. Anti-histamines include, but are not limited to, diphenhydramine, carbinoxamine, clemastine, dimenhydrinate, pryilamine, tripelennamine, chlorpheniramine, brompheniramine, hydroxyzine, cyclizine, meclizine, chlorcyclizine, promethazine, doxylamine, loratadine, and terfenadine. Particular anti-histamines include rhinolast (Astelin), claratyne (Claritin), claratyne D (Claritin D), telfast (Allegra), zyrtec, and beconase.

A TLR agonist will in some embodiments be administered in combination therapy with an agent (other than a TLR agonist) that blocks TGF-β signaling and/or that blocks binding of TGF-β to a TGF-β receptor. Additional agents that block TGF-β signaling and/or that block binding of TGF-β to a TGF-β receptor and that are suitable for use in a subject combination therapy include, but are not limited to, neutralizing antibodies to TGF-β; peptide inhibitors of TGF-β, e.g., as described in U.S. Pat. No. 6,509,318; inhibitors of Smad proteins. See, e.g., U.S. Pat. Nos. 6,365,711; 6,509,318; and 6,277,989.

Combination Therapy with a Therapeutic Agent to Treat Interstitial Lung Disease

In some embodiments, a TLR agonist will in some embodiments be administered as a combination therapy with interferon-gamma (IFN-γ), a corticosteroid, or a combination thereof, for the treatment of interstitial lung disease, e.g., idiopathic pulmonary fibrosis.

IFN-γ may be administered to an individual in a unit dose of from about 70 μg to about 280 μg, from about 100 μg to about 220 μg, or from about 175 μg to about 200 μg. Weight-based dosages of IFN-γ are generally from about 1.0 μg/kg to about 3.5 μg/kg, from about 1.4 to about 3.2 μg/kg, from about 2.0 μg/kg to about 3.0 μg/kg, or from about 2.5 μg/kg to about 2.8 μg/kg. Generally, IFN-γ is administered parenterally, e.g., subcutaneously. In some embodiments, IFN-γ is administered three times per week (tiw).

Corticosteroids, such as prednisone, prednisolone, methyl prednisolone, hydrocortisone, cortisone, dexamethasone, betamethasone, etc. may be administered in an amount of 5 mg-100 mg daily, e.g., from about 5 mg to about 10 mg, from about 10 mg to about 15 mg, from about 15 mg to about 50 mg, from about 50 mg to about 75 mg, or from about 75 mg to about 100 mg, administered orally. Weight-based dosages of a corticosteroid varies from about 100 μg/kg to about 350 μg/kg.

Thus, in some embodiments, the invention provides a method using a combined effective amounts of IFN-γ, and a TLR agonist for the treatment of an interstitial lung disease in a patient, the method comprises administering to the patient a dosage of a TLR agonist containing an amount of from about 1 μg to about 100 mg of drug per dose systemically or directly to the respiratory tract qd, qod, tiw, or biw, or per day for the desired treatment duration; and a dosage of IFN-γ containing an amount of from about 70 μg to about 280 μg of drug per dose of IFN-γ, subcutaneously qd, qod, tiw, or biw, or per day for the desired treatment duration.

In some embodiments, the invention provides a method using a combined effective amounts of IFN-γ, and a TLR agonist for the treatment of an interstitial lung disease in a patient, the method comprises administering to the patient a dosage of a TLR agonist containing an amount of from about 1 μg to about 100 mg of drug per dose systemically or directly to the respiratory tract qd, qod; tiw, or biw, or per day for the desired treatment duration; and a dosage of IFN-γ containing an amount of from about 100 μg to about 220 μg of drug per dose of IFN-γ, subcutaneously qd, qod, tiw, or biw, or per day for the desired treatment duration.

In some embodiments, the invention provides a method using a combined effective amounts of IFN-γ, and a TLR agonist for the treatment of an interstitial lung disease in a patient, the method comprises administering to the patient a dosage of a TLR agonist containing an amount of from about 1 μg to about 100 mg of drug per dose systemically or directly to the respiratory tract qd, qod, tiw, or biw, or per day for the desired treatment duration; and a dosage of IFN-γ containing an amount of from about 175 μg to about 220 μg of drug per dose of IFN-γ, subcutaneously qd, qod, tiw, or biw, or per day for the desired treatment duration.

In some embodiments, the invention provides a method using a combined effective amounts of a corticosteroid, and a TLR agonist for the treatment of an interstitial lung disease in a patient, the method comprises administering to the patient a dosage of a TLR agonist containing an amount of from about 1 μg to about 100 mg of drug per dose systemically or directly to the respiratory tract qd, qod, tiw, or biw, or per day for the desired treatment duration; and a dosage of a corticosteroid containing an amount of from about 100 μg/kg to about 350 μg/kg of drug per dose of corticosteroid orally per day for the desired treatment duration.

In some embodiments, the invention provides a method using a combined effective amounts of a corticosteroid, and a TLR agonist for the treatment of an interstitial lung disease in a patient, the method comprises administering to the patient a dosage of a TLR agonist containing an amount of from about 1 μg to about 100 mg of drug per dose systemically or directly to the respiratory tract qd, qod, tiw, or biw, or per day for the desired treatment duration; and a dosage of a corticosteroid containing an amount of from about 100 μg/kg to about 150 μg/kg of drug per dose of corticosteroid orally per day for the desired treatment duration.

In some embodiments, the invention provides a method using a combined effective amounts of a corticosteroid, and a TLR agonist for the treatment of an interstitial lung disease in a patient, the method comprises administering to the patient a dosage of a TLR agonist containing an amount of from about 1 μg to about 100 mg of drug per dose systemically or directly to the respiratory tract qd, qod, tiw, or biw, or per day for the desired treatment duration; and a dosage of a corticosteroid containing an amount of about 10 mg of drug per dose of corticosteroid orally per day for the desired treatment duration.

In some embodiments, the invention provides a method using a combined effective amounts of IFN-γ, a corticosteroid, and a TLR agonist for the treatment of an interstitial lung disease in a patient, the method comprises administering to the patient a dosage of a TLR agonist containing an amount of from about 1 μg to about 100 mg of drug per dose systemically or directly to the respiratory tract qd, qod, tiw, or biw, or per day for the desired treatment duration; a dosage of IFN-γ containing an amount of from about 100 μg to about 220 μg of drug per dose of IFN-γ, subcutaneously qd, qod, tiw, or biw, or per day for the desired treatment duration; and a dosage of a corticosteroid containing an amount of from about 100 μg/kg to about 350 μg/kg of drug per dose of corticosteroid orally per day for the desired treatment duration.

In some embodiments, the invention provides a method using a combined effective amounts of IFN-γ, a corticosteroid, and a TLR agonist for the treatment of an interstitial lung disease in a patient, the method comprises administering to the patient a dosage of a TLR agonist containing an amount of from about 1 μg to about 100 mg of drug per dose systemically or directly to the respiratory tract qd, qod, tiw, or biw, or per day for the desired treatment duration; a dosage of IFN-γ containing an amount of from about 100 μg to about 220 μg of drug per dose of IFN-γ, subcutaneously qd, qod, tiw, or biw, or per day for the desired treatment duration; and a dosage of a corticosteroid containing an amount of from about 100 μg/kg to about 150 μg/kg of drug per dose of corticosteroid orally per day for the desired treatment duration.

In some embodiments, the invention provides a method using a combined effective amounts of IFN-γ, a corticosteroid, and a TLR agonist for the treatment of an interstitial lung disease in a patient, the method comprises administering to the patient a dosage of a TLR agonist containing an amount of from about 1 μg to about 100 mg of drug per dose systemically or directly to the respiratory tract qd, qod, tiw, or biw, or per day for the desired treatment duration; a dosage of IFN-γ containing an amount of from about 175 μg to about 200 μg of drug per dose of IFN-γ, subcutaneously qd, qod, tiw, or biw, or per day for the desired treatment duration; and a dosage of a corticosteroid containing an amount of from about 100 μg/kg to about 150 μg/kg of drug per dose of corticosteroid orally per day for the desired treatment duration.

In some embodiments, the invention provides a method using a combined effective amounts of IFN-γ, a corticosteroid, and a TLR agonist for the treatment of an interstitial lung disease in a patient, the method comprises administering to the patient a dosage of a TLR agonist containing an amount of from about 1 μg to about 100 mg of drug per dose systemically or directly to the respiratory tract qd, qod, tiw, or biw, or per day for the desired treatment duration; a dosage of IFN-γ containing an amount of from about 175 μg to about 200 μg of drug per dose of IFN-γ, subcutaneously qd, qod, tiw, or biw, or per day for the desired treatment duration; and a dosage of a corticosteroid containing an amount of about 10 mg of drug per dose of corticosteroid orally per day for the desired treatment duration.

In some embodiments, the invention provides a method using a combined effective amounts of IFN-γ, a corticosteroid, and a TLR agonist for the treatment of an interstitial lung disease in a patient, the method comprises administering to the patient a dosage of a TLR agonist containing an amount of from about 1 μg to about 100 mg of drug per dose systemically or directly to the respiratory tract qd, qod, tiw, or biw, or per day for the desired treatment duration; a dosage of IFN-γ containing an amount of about 200 μg of drug per dose of IFN-γ, subcutaneously qd, qod, tiw, or biw, or per day for the desired treatment duration; and a dosage of a corticosteroid containing an amount of about 10 mg of drug per dose of corticosteroid orally per day for the desired treatment duration.

In some embodiments, in any of the above methods, the TLR agonist is a TLR9 agonist, e.g., a therapeutic nucleic acid as described herein. In other embodiments, in any of the above methods, the TLR agonist is a TLR2 agonist. In other embodiments, in any of the above methods, the TLR agonist is a TLR7/8 agonist.

Combination Therapy with a COPD Therapeutic Agent

In some embodiments, a TLR agonist is administered in combination therapy with a known therapeutic agent used in the treatment of COPD. Therapeutic agents used to treat COPD include, but are not limited to, bronchodilators, e.g., isoproterenol, metaproterenol, terbutaline, albuterol, atropine, ipratropium bromide (Atrovent®), Combivent® (ipatropium bromide/salbutamol), Berodual® or Duovent® (fenoterol/ipratropium bromide), and theophylline and its derivatives; corticosteroids/steroids, e.g., beclomethasone, dexamethasone, triamcinolone, and flunisolide; oxygen treatment; antibiotics; and mucolytic agents, e.g., guaifenesin, potassium iodide, and N-acetylcysteine.

Combination Therapy with a Therapeutic Agent for Treating Cystic Fibrosis

In some embodiments, a TLR agonist is administered in combination therapy with a known therapeutic agent used in the treatment of CF. Therapeutic agents used in the treatment of CF include, but are not limited to, antibiotics; anti-inflammatory agents; DNAse (e.g., recombinant human DNAse; pulmozyme; dornase alfa); mucolytic agents (e.g., N-acetylcysteine; Mucomyst™; Mucosil™); decongestants; bronchodilators (e.g., theophylline; ipatropium bromide); and the like.

Individuals Suitable for Treatment

A subject method of treating interstitial lung disease is suitable for treating an individual having interstitial lung disease, regardless of the cause. Suitable subjects include individuals diagnosed with interstitial lung disease. Also suitable for treatment are individuals diagnosed with interstitial lung disease who have failed previous treatment with a therapeutic agent used to treat interstitial lung disease.

Individuals who have a disorder associated with lung fibrosis who are suitable for treatment with a subject method include individuals who have less than about 80%, less than about 75%, less than about 70%, less than about 65%, less than about 60%, less than about 55%, less than about 50%, less than about 45%, or less than about 40%, of the predicted value of a measure of lung function. Measures of lung function include, but are not limited to, forced vital capacity (FVC), forced expiratory volume (FEV), forced expiratory volume in 1 second (FEV₁), FEV/FVC ratio, FEV₁/FVC ratio, and the like. For example, suitable individuals include those exhibiting less than about 75%, less than about 70%, less than about 65%, less than about 60%, less than about 55%, less than about 50%, less than about 45%, or less than about 40%, of the predicted value for FVC. Percent predicted FVC values are based on normal values, which are known in the art. See, e.g., Crapo et al. (1981) Am. Rev. Respir. Dis. 123:659-664. FVC is measured using standard methods of spirometry.

A subject method for treating airway remodeling is useful for treating individuals having, or at risk of developing airway remodeling. Suitable subject include individuals suffering from acute recurrent or chronic asthma.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric. Standard abbreviations are used, e.g., s.c., subcutaneous; i.p., intraperitoneal; min, minute(s); sec, second(s); hr, hour(s); and the like.

### Example 1

### ISS Reduces Fibrosis in a Mouse Model of Airway Remodeling

Methods

Induction of Chronic Pulmonary Eosinophilic Inflammation

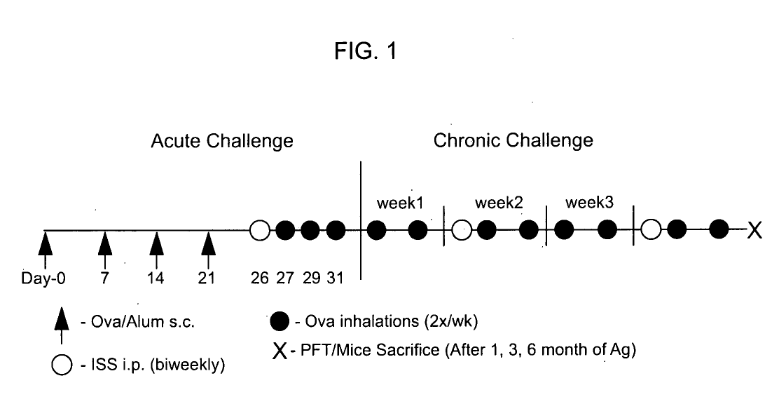

Female BALB/c mice (16 mice/group) (The Jackson Laboratory, Bar Harbor, Me.) were used when they reached 8-10 wk of age. Mice were immunized s.c. on days 0, 7, 14, and 21 with 25 μg of OVA (OVA, grade V; Sigma) adsorbed to 1 mg of alum (Aldrich) in 200 μl normal saline. Intranasal OVA challenges (20 ng/50 μl in PBS) were administered on days 27, 29 and 31 under isoflurane (Vedco, Inc. St Joseph, Mo.) anesthesia. Intranasal OVA challenges were then repeated twice a week for 1, 3, or 6 months (see FIG. 1 for protocol). Age and sex matched control mice were sensitized but not challenged with OVA during the 1, 3, or 6 month study. Mice were sacrificed 24 hours after the final OVA challenge and bronchoalveolar lavage fluid and lungs were analyzed.

Throughout the Examples, therapeutic nucleic acids are referred to as “ISS.” ISS or diluent control was administered intraperitoneally (i.p.) starting 1 day before the first intranasal OVA challenge on day 26, and then continued every other week 1 day before intranasal challenges for 1, 3, or 6 months (see FIG. 1). All animal experimental protocols were approved by the University of California, San Diego Animal Subjects Committees.

Therapeutic Intervention with Immunostimulatory Sequences of DNA (ISS)

Different groups of mice (16 mice/group) were administered i.p. endotoxin-free (<1 ng/mg DNA) phosphorothioate ISS-ODN (5′-TGACTGTGAACGTTCGAGATGA-3′; SEQ ID NO:01) (Trilink, San Diego, Calif.) (100 μg in 100 μl of sterile, endotoxin-free PBS), M-ODN (5′-TGACTGTGAAGGTTGGAGATGA-3′; SEQ ID NO:02) which lacks the CpG motif present in ISS, or diluent control starting 1 day before the first intranasal OVA challenge on day 27, and then continuing every other week 1 day before intranasal challenges for 1, 3, or 6 months. Previous studies in our laboratory have demonstrated that ISS, but not M-ODN, inhibits OVA induced eosinophilic inflammation and airway hyperreactivity when administered 1 day before OVA challenge, and that this inhibitory effect lasts at least 4 weeks.

Determination of Airway Responsiveness to MCh In Vivo

Airway responsiveness was assessed 24 hrs after the final OVA challenge (after 1, 3, or 6 months of repetitive OVA challenges), using a single chamber whole body plethysmograph obtained from Buxco (Troy, N.Y.), as previously described in this laboratory. Broide et al. (1998) J. Immunol. 161:7054. The enhanced pause (Penh) correlates closely with pulmonary resistance measured by conventional two-chamber plethysmography in ventilated mice. Hammelmann et al. (1997) Am. J. Respir. Crit. Care Med. 156:766. In the plethysmograph, mice were exposed for 3 min to nebulized PBS and subsequently to increasing concentrations of nebulized MCh (Sigma, St. Louis, Mo.) in PBS using an Aerosonic ultrasonic nebulizer (DeVilbiss). After each nebulization, recordings were taken for 3 min. The Penh values measured during each 3-min sequence were averaged and are expressed for each MCh concentration as the percentage of baseline Penh values following PBS exposure (Broide et al. supra).

Lung Eosinophil Counts

The sacrificed mice had their tracheas surgically exposed and cannulated with 27-gauge silicon tubing attached to a 23-gauge needle on a 1-ml tuberculin syringe. Following instillation of 800 μl of sterile saline through the trachea into the lung, BALF was withdrawn and cytospun (3 min at 500 rpm) onto microscope slides. Eosinophil counts were performed as previously described (Broide et al. supra).

Quantification of Airway Remodeling

Lungs in the different groups of mice were equivalently inflated with an intratracheal injection of a similar volume of 4% paraformaldehyde solution (Sigma Chemicals, St Louis, Mo.) to preserve the pulmonary architecture. The inflated lungs were embedded in paraffin, stained with either hematoxylin and eosin, Periodic Acid Schiff (PAS), Trichrome stain, or processed for immunohistochemistry.

a) Peribronchial Trichrome Staining

The area of peribronchial trichrome staining in paraffin embedded lung was outlined and quantified using a light microscope (Leica DMLS, Leica Microsystems Inc., NY) attached to an image analysis system (Image-Pro plus, Media Cybernetics, MI). Results are expressed as the area of trichrome staining per μm length of basement membrane of bronchioles 150-200 μm of internal diameter. At least 10 bronchioles were counted in each slide.

b) Lung Immunohistochemical Staining (α-Smooth Muscle Actin, Collagen)

Six μm thick sections of lung from each paraffin block were de-paraffinized with xylene and hydrated in ethanol and phosphate-buffered saline (PBS) pH 7.4. Endogenous peroxidase activity was quenched by incubating lung sections with 0.3% hydrogen peroxide in anhydrous methanol for 5 min. After washing with PBS, the lung sections were incubated with 1% goat serum for 10 min to block non-specific antibody binding.

For immunohistochemical detection of α-smooth muscle actin, the lung sections were incubated overnight at 4° C. with either a primary monoclonal Ab directed against α-smooth muscle actin (Sigma, Saint Louis, Mo.), or as a negative control mouse serum instead of the primary antibody. Immunoreactivity was detected by sequential incubations of lung sections with a biotinylated secondary antibody, followed by peroxidase reagent and AEC chromogen (3-amino-9-ethylcarbazole). The lung sections were briefly incubated with hematoxylin counterstain for 30 seconds, and then mounted with aqueous mounting media. Similar methods were utilized for incubation of anti-collagen primary antibodies (anti-collagen subtypes I, III, V) (Polyscience, Warrington, Pa.), for immunohistochemical detection of collagen. Mouse collagen subtypes were detected using a biotinylated secondary antibody, followed by peroxidase reagent and DAB (3,3′-diaminobenzidine) chromogen (Vector, Burlingame, Calif.).

The area of immunostaining (α-smooth muscle actin or collagen) in each paraffin embedded lung was outlined and quantified using a light microscope attached to an image analysis system (Image-Pro plus). Results are expressed as the area of immunostaining per μm length of basement membrane of bronchioles 150-200 μm of internal diameter. At least 10 bronchioles were counted in each slide.

c) Peribronchial Airway Smooth Muscle Thickness

The thickness of the airway smooth muscle layer was measured using an image analysis system. Lungs which had been fixed in 3% gluteraldehyde and 1% osmium tetroxide were stained with Basic Fuchsin-Toluidine Blue which allowed the best visualization of the peribronchial smooth muscle layer. The thickness of the peribronchial smooth muscle layer (the transverse diameter) was measured from the inner most aspect to the outer most aspect of the circumferential smooth muscle layer. The smooth muscle layer thickness in at least 10 bronchioles of similar size (150-200 μm) were counted on each slide.

d) Quantitation of Airway Mucus Expression

To quantitate the level of mucus expression in the airway, the number of periodic acid Schiffs (PAS) positive and PAS negative epithelial cells in individual bronchioles were counted as previously described in this laboratory. Cho et al. (2001) J. Allergy Clin. Immunol. 108:697. At least 10 bronchioles were counted in each slide. Results are expressed as the % of PAS positive cells/bronchiole which is calculated from the number of PAS positive epithelial cells per bronchus divided by the total number of epithelial cells of each bronchiole.

In addition to quantitating PAS expression, levels of lung Muc 5ac mRNA expression were quantified by RT-PCR. Total cellular RNA was isolated from the lung tissue using TRIZOL reagent (Gibco BRL-Life Technologies, Gaithersburg, Md.) as previously described in this laboratory (Cho et al., supra). The expression of the Muc 5ac gene in lung tissue was carried out with the following sense and anti-sense oligonucleotide sequence primers: sense primer (5′-3′) GGACCAAGTGGTTTGACACTGAC (SEQ ID NO:03), antisense primer CCTCATAGTTGAGGCACATCCCAG (SEQ ID NO:04) (Parmeley and Gendler (1998) J. Clin. Invest. 102:1789). The mouse GAPDH housekeeping gene was used as an internal control. PCR amplification was carried out in a 50 μl reaction volume containing 50 pmol of each primer, 50 mM KCl, 20 mM Tris-HCl, 1.5 mM MgCl₂, 0.2 mM of each dNTP, 1 μl of formamide and 1 unit of Taq polymerase (Gibco, BRL). The reaction mixture was denatured at 95° C. for 5 min, followed by 30 cycles of 95° C. for 30 sec and 60° C. for 30 sec, and extended at 72° C. for 30 sec followed by an extension of 8 min at 72° C. The PCR products (411 bp) were electrophoresed in a 1.5% agarose gel and visualized with ethidium bromide.

Measurement of BAL and Lung Cytokines (TGF-β, IL-13) Associated with Airway Remodeling

The concentrations of TGF-β1 and IL-13 in BAL fluid were assayed by ELISA according to the manufacturer's instructions (R&D Systems). Prior to the TGF-β1 assay, the BAL samples were treated with 2.5 N acetic acid to activate any latent TGF-β1 to immunoreactive TGF-β1 (Khalil (1999) Microbes Infect. 1:1255). Acidified samples were neutralized by 2.7 N NaOH. The TGF-β1 and IL-13 Elisa assays each have sensitivity of 61 pg/ml.

The concentrations of TGF-β1 and IL-13 were also assayed in lung tissue by ELISA (Haeberle et al. (2001) J. Virol. 75:878). Lungs homogenized in lysis buffer (0.5% Triton X-100, 150 mM NaCl, 15 mM Tris, 1 mM CaCl₂, 1 mM MgCl₂) were centrifuged at 10,000×g for 20 min. After the lung supernatant was passaged through a 0.8 μm-pore-size filter, the lung supernatant was assayed for cytokines and protein content. The lung supernatant protein content was assayed using a Micro BCATM protein assay reagent kit (Pierce, Rockford, Ill.) which has a sensitivity of 0.5 μg/ml. Levels of cytokines in lung supernatants were measured by ELISA and results are expressed as pg cytokine/mg protein.

Statistical Analysis

Results in the different groups of mice were compared by ANOVA using the non-parametric Kruskal-Wallis test followed by post-testing using Dunn's multiple comparison of means. All results are presented as mean±SEM. A statistical software package (Graph Pad Prism, San Diego, Calif.) was used for the analysis. P values of <0.05 were considered statistically significant.

Results

Effect of ISS on Airway Responsiveness

Mice sensitized to OVA and challenged with repetitive intranasal administration of OVA developed sustained increases in airway responsiveness to MCh compared to control OVA sensitized mice not repetitively challenged with OVA. The increase in airway responsiveness was evident at all the timepoints studied, e.g. 1 month (p=0.05 vs control), 3 months (p=0.05 vs control) or 6 months (p=0.05 vs control). Systemic administration of ISS significantly reduced airway responsiveness to MCh in mice repetitively challenged with OVA compared to untreated mice repetitively challenged with OVA at 1 month (p=0.05), 3 months (p=0.05), and 6 months (p=0.05). In pilot experiments M-ODN (similar to no treatment) did not inhibit OVA induced airway responsiveness, eosinophilic inflammation, or features of airway remodeling.

Effect of ISS on BAL Eosinophils

The absolute number of BAL eosinophils in mice sensitized to OVA and repetitively challenged with OVA was significantly greater than in control non-OVA challenged mice at 1 month (45.9±5.55×10³ vs 0.3±0.1×10³ BAL eosinophils)(p=0.0001), 3 months (34.9±5.8×10³ vs 0.10±0.03×10³ BAL eosinophils)(p=0.0001), and 6 months (8.9±2.3×10³ vs 0.1±0.1×10³ BAL eosinophils) (p=0.0001). Although the number of BAL eosinophils in mice repetitively challenged with OVA were still significantly increased at 6 months compared to control non-OVA challenged mice (8.9×10³ vs 0.1×10³ BAL eosinophils) the number of BAL eosinophils in mice repetitively challenged with OVA were less at 6 months (8.9×10³ BAL eosinophils) compared to mice repetitively challenged with OVA at 1 month (45.9×10³ BAL eosinophils) and 3 months (34.9×10³ BAL eosinophils).

Systemic administration of ISS prior to initiation of repetitive OVA challenges significantly reduced the absolute number of BAL eosinophils compared to untreated mice challenged repetitively with OVA at 1 month (16.87±4.58×10³ vs 45.9±5.55×10³ BAL eosinophils)(p=0.001), and 3 months (13.3±3.9×10³ vs 34.9±5.8×10³ BAL eosinophils)(p=0.001), while the reduction at 6 months did not reach statistical significance (4.1±0.7×10³ vs 8.9±2.3×10³ BAL eosinophils) (p=0.15).

ISS Reduces Peribronchial Fibrosis

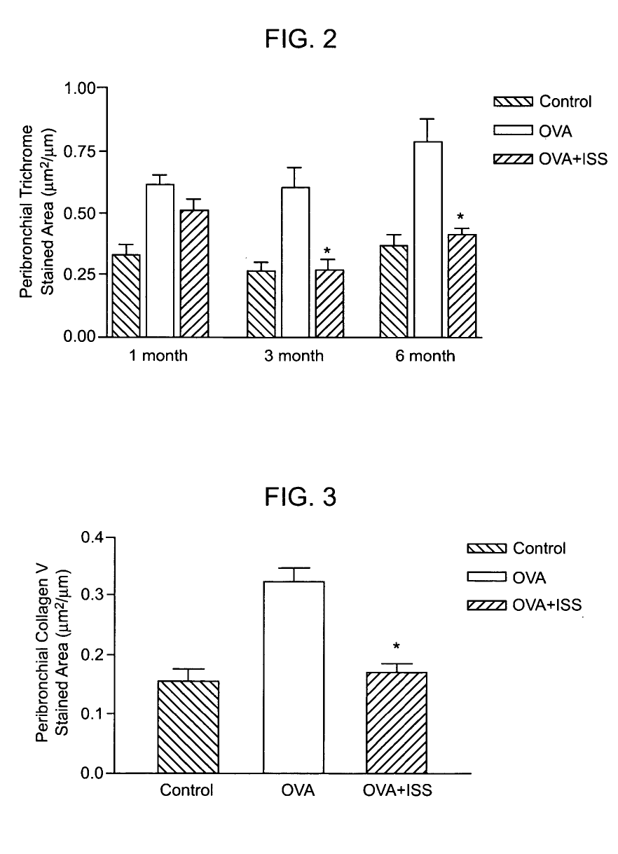

We used two methods to quantitate peribronchial fibrosis, namely the area of peribronchial trichrome staining, and the area of peribronchial immunostaining with anti-collagen V Ab (expressed as the stained area in μm²/μm circumference of bronchiole). The area of peribronchial trichrome stain in mice which were repetitively challenged with OVA was significantly greater than in control non-OVA challenged mice at 3 months (0.60±0.08 vs 0.26±0.03 μm²/μm circumference of bronchiole)(p=0.004), and 6 months (0.79±0.09 vs 0.36±0.04 μm²/μm circumference of bronchiole)(p=0.0001), while the increase at 1 month did not reach statistical significance (0.58±0.04 vs 0.32±0.04 μm²/μm circumference of bronchiole)(p=0.12), (FIG. 2).

FIG. 2: Mice repetitively challenged with OVA for 3 months (p=0.004, OVA vs control), or 6 months (p=0.0001, OVA vs control), but not 1 month (p=ns, OVA vs control), developed increased peribronchial trichome staining compared to control non-OVA challenged mice. Systemic administration of ISS significantly reduced levels of peribronchial trichrome staining in mice challenged repetitively with OVA, compared to untreated mice challenged repetitively with OVA for 3 months (p=0.0003, vs ISS+OVA vs OVA), or 6 months (p=0.0001, ISS+OVA vs OVA).

Systemic administration of ISS to mice repetitively challenged with OVA significantly reduced the area of trichrome staining compared to untreated mice repetitively challenged with OVA at 3 months (0.27±0.03 vs 0.60±0.08 μm²/μm circumference of bronchiole)(p=0.0003), and 6 months (0.40±0.02 vs 0.79±0.09 μm²/μm circumference of bronchiole) (p=0.0001)(FIG. 2). Pre-treatment with ISS did not significantly inhibit levels of trichrome staining at 1 month in mice repetitively challenged with OVA compared to untreated mice repetitively challenged with OVA (0.58±0.04 vs 0.55±0.06 μm²/μm circumference of bronchiole)(p=NS).

The area of peribronchial trichrome staining noted in mice repetitively challenged with OVA and pre-treated with ISS for 3 months was reduced to levels of background peribronchial trichrome staining noted in non-OVA challenged control mice (0.27±0.04 vs 0.27±0.03 μm²/μm circumference of bronchiole)(FIG. 2). Similar beneficial effects of ISS on reducing peribronchial trichrome staining to levels of non-OVA challenged mice were also noted in mice treated with ISS for 6 months (0.40±0.02 vs 0.36±0.04 μm²/μm circumference of bronchiole)(FIG. 2).

Effect of ISS on Peribronchial Collagen Immunostaining

Pilot immunostaining studies of remodeled airways with anti-collagen I, III, and V Abs demonstrated that anti-collagen staining with the anti-collagen V Ab was reproducibly detected, whereas staining with anti-collagen I and III Abs was more variable in the lungs of mice repetitively challenged with OVA. Therefore we quantitated only anti-collagen V immunostaining. The area of peribronchial collagen V immunostaining in mice which were repetitively challenged with OVA was significantly greater than in control non-OVA challenged mice at 3 months (0.32±0.02 vs 0.15±0.02 μm²/μm circumference of bronchiole)(p=0.0001)(FIG. 3).

FIG. 3: Mice repetitively challenged with OVA for 3 months (p=0.0001, OVA vs control) developed increased peribronchial collagen V immunostaining compared to control non-OVA challenged mice. Systemic administration of ISS significantly reduced levels of peribronchial collagen V immunostaining in mice challenged repetitively with OVA, compared to untreated mice challenged repetitively with OVA for 3 months (p=0.0001, ISS+OVA vs OVA).

Systemic administration of ISS to mice repetitively challenged with OVA significantly reduced the area of collagen V immunostaining compared to untreated mice repetitively challenged with OVA at 3 months (0.17±0.02 vs 0.32±0.02 μm²/μm circumference of bronchiole)(p=0.0001)(FIG. 3).

Effect of ISS on Peribronchial Smooth Muscle Layer Thickness

The thickness of the peribronchial smooth muscle layer (measured in μm) in mice repetitively challenged with OVA was significantly greater than in control non-OVA challenged mice at 1 month (13.6±0.4 vs 5.3±0.5 μm)(p=0.0001), 3 months (14.3±0.7 vs 8.9±0.6 μm)(p=0.0001), and 6 months (15.0±0.4 vs 8.3±0.4 μm)(p=0.0001).

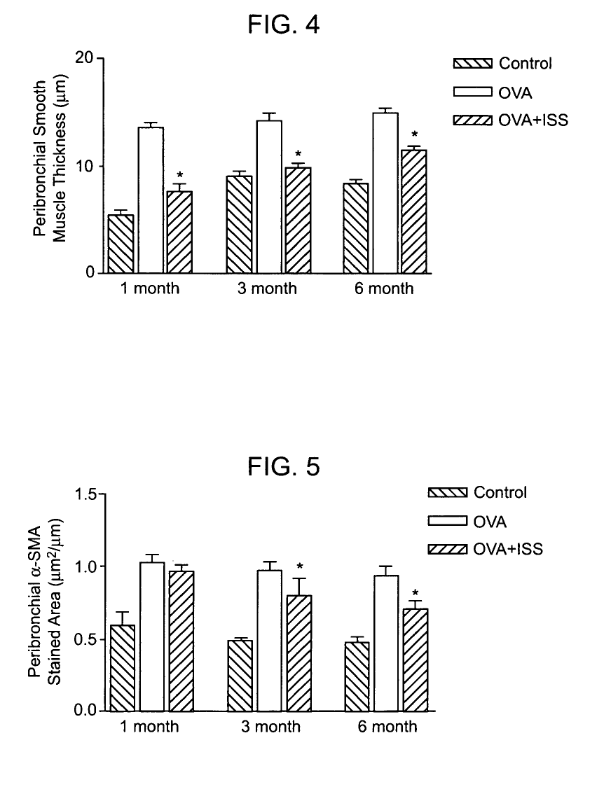

Systemic administration of ISS significantly reduced the peribronchial smooth muscle layer thickness in mice repetitively challenged with OVA compared to untreated mice repetitively challenged with OVA at 1 month (7.5±0.8 vs 13.6±0.4 μm)(p=0.0001), 3 months (9.8±0.5 vs 14.3±0.7 μm)(p=0.0001), and 6 months (11.5±0.3 vs 15.0±0.4 μm) (p=0.0001) (FIG. 4).

FIG. 4: Mice repetitively challenged with OVA for 1 month (p=0.0001, OVA vs control), 3 months (p=0.0001, OVA vs control), or 6 months (p=0.0001, OVA vs control), developed increased thickness of the peribronchial smooth muscle layer compared to control non-OVA challenged mice. Systemic administration of ISS significantly reduced the peribronchial smooth muscle layer thickness in mice challenged repetitively with OVA, compared to untreated mice challenged repetitively with OVA for 1 month (p=0.0001, vs ISS+OVA vs OVA), 3 months (p=0.0001, vs ISS+OVA vs OVA), or 6 months (p=0.0001, ISS+OVA vs OVA).

Effect of ISS on Area of Peribronchial Myofibroblast α-Smooth Muscle Actin Immunostaining

The area of peribronchial myofibroblast α-smooth muscle actin immunostaining was quantified by image analysis and expressed as the stained area in μm²/μm circumference of a bronchiole. The area of peribronchial α-smooth muscle actin immunostaining in mice repetitively challenged with OVA was significantly greater than in control non-OVA challenged mice at 1 month (0.88±0.06 vs 0.60±0.09 μm²/μm circumference of bronchiole)(p=0.004), 3 months (0.97±0.06 vs 0.49±0.02 μm²/μm circumference of bronchiole)(p=0.0001), and 6 months (0.94±0.07 vs 0.48±0.03 μm²/μm circumference of brorichiole) (p=0.0001)(FIG. 5).

FIG. 5: Mice repetitively challenged with OVA for 1 month (p=0.004, OVA vs control), 3 months (p=0.0001, OVA vs control), or 6 months (p=0.0001, OVA vs control), developed an increase in the area of peribronchial myofibroblast immunostaining compared to control non-OVA challenged mice. Systemic administration of ISS significantly reduced the area of peribronchial myofibroblast immunostaining in mice challenged repetitively with OVA, compared to untreated mice challenged repetitively with OVA for 3 months (p=0.0001, ISS+OVA vs OVA), or 6 months (p=0.0001, ISS+OVA vs OVA), but not for 1 month (p=ns, ISS+OVA vs OVA).

Systemic administration of ISS prior to repetitive OVA challenges significantly reduced the area of α-smooth muscle actin immunostaining compared to untreated mice challenged repetitively with OVA at 3 months (0.80±0.11 vs 0.97±0.06 μm²/μm circumference of bronchiole)(p=0.0001), and 6 months (0.72±0.05 vs 0.94±0.06 μm²/μm circumference of bronchiole) (p=0.001)(FIG. 5). Pre-treatment with ISS for 1 month did not significantly inhibit the area of α-smooth muscle actin immunostaining in mice repetitively challenged with OVA, compared to untreated mice repetitively challenged with OVA at 1 month (p=NS).

Effect of ISS on BAL Fluid TGF-β1 and IL-13 Levels

As both TGF-β1 and IL-13 are able to induce peribronchial fibrosis, we measured levels of these cytokines in BAL fluid and lung tissue. Levels of BAL TGF-β1 were significantly increased in mice exposed to repetitive OVA challenge compared to control non-OVA challenged mice (156±28 vs 56±36 pg/ml TGF-Pl)(p=0.03). ISS significantly reduced levels of BAL TGF-β1 in mice exposed to repetitive OVA challenge compared to untreated mice challenged repetitively with OVA (40±19 vs 156±28 pg/ml TGF-β1)(p=0.05).

Similarly levels of lung TGF-β1 were significantly increased in mice exposed to repetitive OVA challenge compared to control non-OVA challenged mice (1946±261 vs 664±75 pg TGF-β1/mg lung protein)(p=0.003). ISS significantly reduced levels of lung TGF-β1 in mice exposed to repetitive OVA challenge compared to untreated mice challenged repetitively with OVA (939±171 vs 1946±261 pg TGF-β1/mg lung protein)(p=0.004).

Levels of BAL IL-13 were also significantly increased in mice exposed to repetitive OVA challenge compared to control non-OVA challenged mice (4,189±731 vs 1597±590 pg/ml IL-13)(p=0.01). Although ISS reduced levels of BAL IL-13 in mice exposed to repetitive OVA challenge compared to untreated mice challenged repetitively with OVA, this reduction was not statistically significant (2,941±696 vs 4,189±731 pg/ml IL-13)(p=NS). ISS did not significantly reduce levels of lung IL-13 in mice exposed to repetitive OVA challenge compared to untreated mice challenged repetitively with OVA.

Effect of ISS on Airway Mucus Expression

The % of airway epithelium which stained positive with PAS in mice repetitively challenged with OVA was significantly greater than in control non-OVA challenged mice at 1 month (13.1±1.6 vs 0.1±0.01%)(p=0.0001), 3 months (22.1±2.8 vs 0.1 ±0.01%)(p=0.0001), and 6 months (22.9±3.4 vs 0.2±0.2%) (p=0.0005).

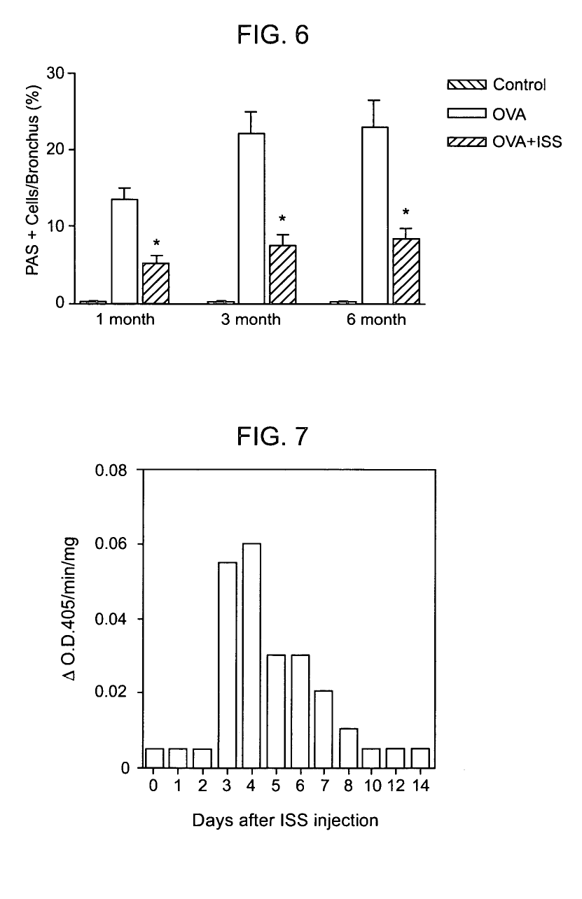

Systemic administration of ISS in mice repetitively challenged with OVA significantly reduced the % of airway epithelium staining positively with PAS compared to untreated mice repetitively challenged with OVA at 1 month (5.1±0.9, vs 13.1±1.6%)(p=0.0005), 3 months (7.4±1.3 vs 22.1±2.8%)(p=0.0003), and 6 months (8.4±1.2 vs 22.9±3.4%) (p=0.02) (FIG. 6).

FIG. 6: Mice repetitively challenged with OVA for 1 month (p=0.0001, OVA vs control), 3 months (p=0.0001, OVA vs control), or 6 months (p=0.0005, OVA vs control), developed increased PAS staining of airway epithelium compared to control non-OVA challenged mice. Systemic administration of ISS significantly reduced the PAS staining of airway epithelium in mice challenged repetitively with OVA, compared to untreated mice challenged repetitively with OVA for 1 month (p=0.0005, vs ISS+OVA vs OVA), 3 months (p=0.0003, vs ISS+OVA vs OVA), or 6 months (p=0.02, ISS+OVA vs OVA).

ISS also significantly inhibited lung Muc 5ac mRNA expression as assessed by RT-PCR in mice repetitively challenged with OVA as compared to untreated mice repetitively challenged with OVA (FIG. 7).

FIG. 7: Repetitive OVA challenge for 3 months induced significant levels of lung Muc 5 ac as assessed by RT-PCR (OVA; lanes 1-4). ISS significantly inhibited Muc 5ac expression in mice challenged repetitively for 3 months with OVA (ISS+OVA; lanes 5-8) compared to untreated mice challenged repetitively with OVA for the same time period (OVA; lanes 1-4). Mice not challenged with OVA (Control; lanes 9-12) have minimal expression of Muc 5ac. The mouse housekeeping gene mGAPDH demonstrates equivalent loading of lanes.

### Example 2

### ISS Inhibit Mast Cell Growth and Function

Peribronchial mast cells are infrequently observed in the lungs of naïve mice (0±0 mast cells per large, medium, and small airways, n=8 mice), or in OVA sensitized and acute OVA challenged mice (0.05±0.04 mast cells per large airway and 0±0 mast cells per medium and small airways, n=8 mice). However, repetitive OVA challenge for 1 to 6 months induced a significant increase in airway mast cell numbers. Repetitive OVA challenge significantly increases the number of mast cells in the large airways (10.5±1.0 mast cells per airway), medium sized airways (4.3±0.9), and small airways (1.7±0.3) after 1 month. Similarly, repetitive OVA challenge increased the number of mast cells in large, medium, and small airways at 3 and 6 months. The mean number of mast cells in large airways (10.5 mast cells) was greater than the number of mast cells in medium (4.3) mast cells) and small (1.7 mast cells) sized airways.

ISS-treated mice had a significantly lower number of airway mast cell in the large airways after repetitive OVA challenge for 1 month (6.8±0.8 vs 10.5±1.0 mast cells per airway) (p=0.006), 3 months (5.5±0.8 vs 9.5±1.1 mast cells per airway (p=0.02), and 6 months (2.4±0.6 vs 7.3±0.6 mast cells per airway) (p=0.0001). Similar results were noted in medium and small sized airways.

### Example 3

### Toll-Like Receptor Ligands Inhibit TGF-β Signaling in Lung Tissue

TGF-β has anti-inflammatory properties and inhibits macrophages, natural killer cells, and T-cell functions. In addition to the anti-inflammatory activities, TGF-β may play a central role in tissue repair and tissue fibrosis. The integrin αᵥβ₆ expressed on epithelial cells, such as those that line the lungs, activates TGF-β through binding of latency-associated peptide (LAP). The data presented below demonstrate that administration of a TLR agonist in vivo inhibits TGF-β signaling in lung tissue.

Twenty μg ISS was injected intravenously (i.v.) into C57BL/6 mice. At 0 hours, 2 hours, 4 hours, 8 hours, 16 hours, and 24 hours after injection, mice were killed and total RNA was isolated from lung. Transcription levels of IGFβ₆ (β₆ integrin) was analyzed by reverse transcription-polymerase chain reaction (RT-PCR). TNFα, IDO (2,3-indoleaminedioxygenase) and TGFβ1 were used as controls. The data show that transcription of the IGFβ₆ was suppressed in less than 2 hours and that the suppression lasted for at least 24 hours. Transcription of the other subunit of ITGαᵥβ₆ (or αᵥβ₆ integrin), i.e., ITGαᵥ, was also analyzed by RT-PCR. Levels of ITGαᵥ mRNA were also suppressed in less than 2 hours by i.v. ISS administration, and the suppression lasted at least 24 hours.

Western blot analysis was performed to confirm the suppression of ITGαᵥβ₆ by ISS. Twenty μg ISS was injected i.v. into C57 BL/6 mice. At 0 hours, 2 hours, 4 hours, 8 hours, 16 hours, and 24 hours after injection, mice were killed and lungs removed. Isolated lungs were homogenized in lysis buffer and 20 μg each lung crude extract was loaded onto 10-20% tricine sodium dodecyl sulfate-polyacrylamide gels (SDS-PAG) and subjected to SDS-PAG electrophoresis (SDS-PAGE). After electrophoresis, proteins were transferred onto a PVDF membrane and incubated with antibody to αᵥ and to β₆ integrin subunits. The results indicated that protein levels of both integrin subunits were suppressed after ISS administration.

The long-term effect of ISS on gene expression in lung tissue was investigated. Twenty μg ISS was injected i.v. into C57 BL/6 mice. At 0 days, 1 day, 2 days, 3 days, 4 days, and 5 days post injection, mice were killed and total RNA was isolated from lung tissue. Transcription levels were analyzed by RT-PCR. The results showed that the down-regulation of transcription of IGFαᵥ and ITGβ₆ genes lasted 2 days following administration of ISS. The suppression of TGF-β activity was observed indirectly by the induction of MMP-12 (elastase) gene. Under normal conditions, MMP-12 expression is suppressed by TGF-β. The data showed that when TGF-β is suppressed, MMP-12 mRNA levels are increased. In contrast, no change in mRNA levels of MMP-9 (gelatinase) was observed.

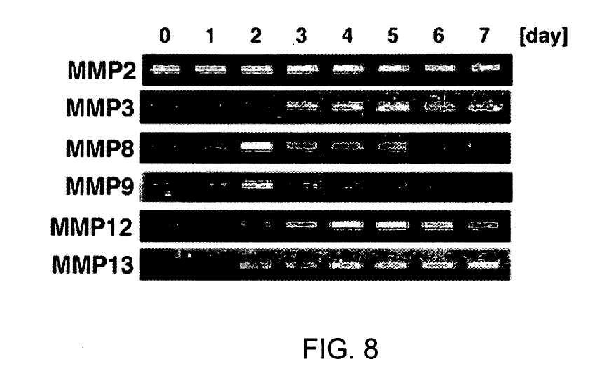

Induction of enzymatic activity of various matrix metalloproteinases (MMPs) by ISS in lung tissue was also observed. Twenty μg ISS was injected i.v. into C57 BL/6 mice. At different time points post-injection (e.g., 0 days, 1 day, 2 days, 3 days, 4 days, 5 days, 6 days, 7 days, 8 days, 10 days, 12 days, and 14 days post-injection), mice were killed and lung tissues were homogenized in 50 mM Tris-HCl buffer, pH 7.5. MMP-12 proteolytic activity was measured using a BIOMOL MMP-12 calorimetric assay kit. Other MMP enzyme activity was measured using assays that are standard in the field. The results are shown in FIG. 8. The results showed that MMP-12 enzymatic activity correlated with MMP-12 mRNA levels, and is induced from day 3 to day 6 after ISS injection. ISS induces enzymatic activity of MMP3, MMP8, MMP9, MMP12, and MMP13.

The kinetics of suppression of gene expression by ISS was analyzed. The results, from both conventional RT-PCR and real-time quantitative PCR, showed that suppression of transcription of ITGαᵥ and ITGβ₆ integrin subunit genes proceeds rapidly. The level of ITGαᵥ mRNA dropped to 20% of the initial levels within 30 minutes after i.v. injection of ISS.

Suppression of αᵥβ₆ gene transcription following i.v. injection of OSS was evaluated in various organs and tissues (colon, heart, intestine, liver, lung, and spleen). The data showed that suppression of αᵥβ₆ gene transcription occurred in lung, but not in the other organs and tissues analyzed.

MyD88 is a component of the TLR signaling pathway. It was shown that down-regulation of ITGβ₆ gene transcription by ISS in lung tissue is dependent on MyD88. ISS suppressed ITGβ₆ gene transcription in normal control mice, but not in MyD88^{-/-} mice.

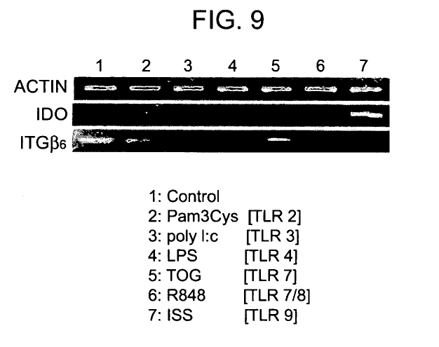

Suppression of ITGβ₆ gene transcription in lung tissue by a variety of TLR ligands was examined. Six different TLR ligands (Pam₃Cys, 25 μg; polyI:C, 25 μg; LPS, 5 μg; TOG, 2 mg; R848, 18 μg; and ISS, 20 μg) were individually injected i.v. into B6 mice. After 24 hrs, mice were sacrificed and total RNA was isolated form lung tissue, and the level of ITGβ₆ gene transcription in lung tissue was analyzed by RT-PCR. The results are shown in FIG. 9. Each TLR agonist inhibited ITGβ₆ gene transcription in lung tissue.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A method for treating airway remodeling in an individual suffering from chronic asthma, the method comprising administering to the individual an effective amount of a toll-like receptor (TLR) agonist, wherein at least one pathological parameter associated with airway remodeling is reduced.

2. The method of claim 1, wherein the TLR is TLR9, and the TLR agonist is a nucleic acid that comprises the sequence 5′ CG 3′.

3. A method for treating interstitial lung fibrosis in an individual, the method comprising administering to the individual an effective amount of a toll-like receptor (TLR) agonist in an amount effective to treat the disorder.

4. The method of claim 3, wherein the lung fibrosis is associated with a condition selected from idiopathic pulmonary fibrosis, sarcoidosis, chronic obstructive pulmonary disease, cystic fibrosis, chronic exposure to an irritant, chronic viral infection of the lungs, chronic mycoplasma infection of the airways, and chronic bacterial infection of the lungs.

5. The method of claim 3, wherein the TLR is TLR9, and the TLR agonist is a nucleic acid that comprises the sequence 5′ CG 3′.

6. The method of claim 2 or claim 5, wherein the nucleic acid comprises a nucleotide sequence selected from 5′-purine-purine-cytosine-guanine-pyrimidine-pyrimidine-3′, 5′-purine-TCG-pyrimidine-pyrimidine-3′, and 5′-(TGC)ₙ-3′, where n≧1.

7. The method of claim 2 or claim 5, wherein the nucleic acid comprises a nucleotide sequence of the formula 5′-TCG-N-N-3′; where n is any base.

8. The method of claim 2 or claim 5, wherein the nucleic acid comprises a nucleotide sequence of the formula 5′ Nₘ-(TCG)n-Nₚ-3′, wherein N is any nucleotide, wherein m is zero, one, two, or three, wherein n is any integer that is 1 or greater, and wherein p is one, two, three, or four.

9. The method of claim 2 or claim 5, wherein the nucleic acid comprises a nucleotide sequence of the formula 5′ Nₘ-(TCG)ₙ-Nₚ-3′, where N is any nucleotide, wherein m is zero to 5, wherein n is any integer that is 1 or greater, wherein p is four or greater, and wherein the sequence N-N-N-N comprises at least two CG dinucleotides that are either contiguous with each other or are separated by one nucleotide, two nucleotides, or three nucleotides.

10. The method of claim 1 or claim 3, wherein the TLR agonist is administered to the respiratory tract of the individual.

11. The method of claim 1 or claim 3, wherein the TLR agonist is administered intranasally.

12. The method of claim 1 or claim 3, wherein the TLR agonist is administered systemically.

13. The method of claim 1 or claim 3, further comprising administering an effective amount of an anti-inflammatory agent.

14. The method of claim 3, further comprising administering an effective amount of a corticosteroid.

15. The method of claim 11, wherein the corticosteroid is predisolone.

16. The method of claim 3, further comprising administering an effective amount of IFN-γ.

17. The method of claim 3, further comprising effective amounts of a corticosteroid and IFN-γ.

18. A pharmaceutical formulation for treatment of lung fibrosis, comprising:
a therapeutically effective amount of toll-like receptor (TLR) agonist; and
a flowable formulation suitable for delivery by inhalation.

19. The pharmaceutical formulation of claim 18, wherein the TLR agonist is formulated with a fluid carrier and a propellant.

20. The pharmaceutical formulation of claim 18, wherein the TLR agonist is formulated in an aqueous or an ethanolic solution.

21. The pharmaceutical formulation of claim 18, wherein the TLR agonist is in a dry powder formulation.

22. The pharmaceutical formulation of claim 18, wherein the TLR agonist is aerosolized to create an aerosol.

23. A package for use in treating lung fibrosis, comprising a container having therein a flowable formulation suitable for delivery by inhalation, the formulation comprising a pharmaceutically active toll-like receptor agonist.


SEQUENCE LISTING (5 sequences)

SEQ ID NO: 1 - DNA, 22 bp, Artificial Sequence
  synthetic DNA
tgactgtgaacgttcgagatga

SEQ ID NO: 2 - DNA, 22 bp, Artificial Sequence
  synthetic DNA
tgactgtgaaggttggagatga

SEQ ID NO: 3 - DNA, 23 bp, Artificial Sequence
  synthetic primer
ggaccaagtggtttgacactgac

SEQ ID NO: 4 - DNA, 24 bp, Artificial Sequence
  synthetic primer
cctcatagttgaggcacatcccag

SEQ ID NO: 5 - DNA, 24 bp, Artificial Sequence
  synthetic DNA
tcgtcgttttgtcgttttgtcgtt